(19) United States
(12) Patent Application Publication
(10) Pub. No.: US 2006/0253936 A1
(43) Pub. Date: Nov. 9, 2006

(54) Novel constructs and their use in metabolic pathway engineering

(75) Inventors: Lu Liu, Redwood City, IA (US); Genhai Zhu, San Jose, CA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: Pioneer Hi-Bred International Inc., Johnston, IA (US)

(21) Appl. No.: 11/485,024

(22) Filed: Jul. 12, 2006

Related U.S. Application Data
(63) Continuation of application No. 09/932,254, filed on Aug. 16, 2001.
(60) Provisional application No. 60/227,719, filed on Aug. 24, 2000.

(51) Int. Cl. A01H 1/00; C12N 9/10; C12N 15/82; C12N 5/04

(52) U.S. Cl. 800/288; 435/468; 435/419; 435/193

(57) ABSTRACT

The present invention relates generally to methods and techniques for the expression of metabolic pathways, novel gene fusion constructs encoding multi-functional enzymatic domains, and related hybrid proteins.

### CROSS-REFERENCE TO RELATED APPLICATIONS

This application claims priority to U.S. Ser. No. 09/932,254 filed Aug. 16, 2001, which claimed priority to U.S. Ser. No. 60/227,719, filed Aug. 24, 2000, the disclosures of which are incorporated by reference for all purposes.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

This invention pertains to the field of molecular biology, more particularly to methods of creating gene fusion constructs encoding two or more fused enzymatic domains.

### BACKGROUND OF THE INVENTION

Metabolic pathways are, in essence, collections of enzymatic activities which, when performed in a certain order, lead from a starting material to a desired final product. In some circumstances, the metabolic pathway is a synthesis procedure; in others, it is a degradative process. The synthesis and coordination of the enzyme components of metabolic pathways is relatively straight-forward in the mostly uncompartmentalized cellular environment of prokaryotic cells. Transcription and translation in prokaryotes are coupled, both spatially and temporally. Since prokaryotic cells do not have a membrane-bound nucleus, transcription and translation are not compartmentalized as in a eukaryote, and these processes take place in the same cellular location, the cytoplasm. However, eukaryotes are more compartmentalized in their cellular structure. Establishing and implementing a new metabolic pathway into a desired compartment of a eukaryotic system, such as a plant, for example, is more difficult than establishing a comparable metabolic pathway in a prokaryotic system, due to, for example, the additional hurdles of coordination of transcriptional and translational events for multiple proteins, intracellular compartmentalization issues, and the use of multiple promoter, initiation and termination systems. Accordingly, new methods for facilitating metabolic pathway engineering in organisms, particularly eukaryotes, would be desirable.

The present invention provides methods and compositions for the expression of metabolic pathways and pathway components in, e.g., eukaryotes such as plant systems.

### SUMMARY OF THE INVENTION

Engineering of metabolic pathways can be used both for the production of novel metabolites, as well as for the enhancement or augmentation of current protocols for production of existing metabolites. The transfer of metabolic pathways among species also provides novel ways to produce desired metabolites in specific hosts. For example, transfer of a bacterial metabolic pathway for production of a chemical compound into plant systems enables production of this compound in an alternative and potentially economically competitive manner, as compared to traditional chemical syntheses or bacterial fermentation. Alternatively, transfer and expression of the metabolic pathway components and the resulting metabolite(s) can confer a desired trait upon the recipient system.

Accordingly, the present invention provides methods for producing a modified gene fusion construct, including cojoining two or more (and often, three or more) nucleic acid sequences that encode two or more enzymatic domains, where at least one of the nucleic acid sequences has been modified (for example, mutated, shuffled, or otherwise altered) as compared to an originally-determined (i.e., unmodified) sequence. The modified nucleic acid sequence can be modified prior to cojoining the sequence to the second nucleotide sequence, or it can be modified after the sequences are cojoined. Optionally, the modified nucleic acid sequence has undergone recursive recombination to produce the modification in the sequence. The nucleic acid sequences can be various forms of deoxyribonucleic acid (for example, genomic DNA, cDNA, sense-strand sequences, antisense-strand sequences, recombinant DNA, shuffled DNA, modified DNA, or DNA analogs). Alternatively, the nucleic acid sequences can be ribonucleic acid (including, but not limited to, genomic RNA, messenger RNA, catalytic RNA, sense-strand sequences, antisense-strand sequences, recombinant RNA, shuffled RNA, modified RNA, or RNA analogs). The nucleic acid sequences can be joined together directly, or they can be separated by one or more nucleotide linker sequences. Nucleotide linker sequences of the invention typically range in length from about three to about three hundred nucleotides, but can in some cases be longer. Optionally, the nucleotide linker sequences include introns, restriction enzyme sites, intein-encoding sequences, and/or sequences that encode cleavable peptide regions. As with the nucleotide sequences encoding the enzymatic domains, the nucleotide linker sequences can be modified, for example, by mutation, shuffling, or other alterations. In addition, one or more transcription regulatory sequences (for example, promoters or enhancers) can be incorporated into the modified gene fusion construct. The modified gene fusion construct can be further introduced into a eukaryotic system, for example, a plant system.

The nucleic acids incorporated into the modified gene fusion constructs of the present invention can be derived from a single metabolic pathway, or they can be derived from two or more distinct metabolic pathways (e.g., to produce a novel metabolic pathway). In addition, the nucleic acids incorporated into the gene fusion constructs can be derived from a single source or species, or they can originate from multiple sources or species. In one embodiment of the present invention, the enzymatic domains encoded by the two or more nucleic acid sequences are derived from the enzymes phytoene synthase, phytoene desaturase, and/or beta-cyclase. In an alternative embodiment of the present invention, the enzymatic domains encoded by the two or more nucleic acid sequences are derived from the enzymes diaminobutyric acid aminotransferase, diaminobutyric acid acetyltransferase, and ectoine synthase. In another embodiment of the present invention, the enzymatic domains encoded by the two or more nucleic acid sequences are derived from the enzymes beta-ketothiolase, D-reductase, and poly(hydroxyalkanoate) synthase. In a further embodiment of the present invention, the enzymatic domains encoded by the two or more nucleic acid sequences are derived from the following classes of enzymes: ketosynthase-acyltransferases, chain length factors, acyl carrier proteins, and cyclases. Furthermore, the present invention provides modified fusion constructs, vectors comprising the modified gene constructs, hybrid proteins, and transgenic systems, such as transgenic plant systems.

The present invention also provides methods for producing a gene fusion construct by cojoining two or more heterologous nucleic acid sequences that participate in the same metabolic pathway, wherein at least one of the cojoined nucleic acid sequences is derived from a eukaryote and another cojoined nucleic acid sequence is derived from either a different species of eukaryote or from a prokaryote. The nucleic acid sequences of interest in the previously described method of producing a modified fusion construct can be used in methods employing two or more heterologous nucleic acid sequences derived from two or more eukaryotes or from at least one prokaryote and at least one eukaryote. In addition, similar nucleotide linker sequences and transcription regulatory elements can be used. The methods can further include the step of introducing the modified gene fusion construct into a prokaryotic or eukaryotic system, for example, a plant system. Furthermore, the present invention provides gene fusion constructs, vectors comprising the gene fusion constructs, hybrid proteins, and transgenic systems, such as transgenic plant systems.

The present invention also provides methods for producing a gene fusion construct by cojoining two or more nucleic acid sequences encoding heterologous enzymatic domains, wherein at least one of the enzymatic domains is derived from a plant. The plant enzymatic domains can be derived from, for example, enzymes involved in the biosynthesis of carotenoids. The nucleic acid sequences can be various forms of deoxyribonucleic acid or ribonucleic acid, as described for the methods for producing a modified gene fusion construct. In addition, similar nucleotide linker sequences and transcription regulatory elements are optionally used. The methods can further include the step of introducing the gene fusion construct into a biological system, for example, a prokaryotic system or a eukaryotic system. Furthermore, the present invention provides gene fusion constructs, vectors comprising the gene fusion constructs, hybrid proteins, and transgenic biological systems, such as transgenic bacterial, fungal, or plant system.

The present invention also provides methods for expressing a plurality of enzyme activities in a biological system, for example, a prokaryotic system or a eukaryotic system. The methods include the step of introducing any one or more of the aforementioned gene constructs into a biological system. The nucleic acid sequences generally encode proteins that can participate in a metabolic pathway, wherein the pathway can, but need not occur in nature, e.g., in the case where a novel metabolic pathway is created by combining enzymatic domains that do not normally function in the same pathway in nature. In one embodiment of the present invention, the enzymatic domains encoded by the nucleic acid sequences are derived from the enzymes phytoene synthase, phytoene desaturase, and/or beta-cyclase. In an alternative embodiment of the present invention, the enzymatic domains encoded by the nucleic acid sequences are derived from the enzymes diaminobutyric acid aminotransferase, diaminobutyric acid acetyltransferase, and ectoine synthase. In another embodiment of the present invention, the enzymatic domains encoded by the nucleic acid sequences are derived from the enzymes beta-ketothiolase, D-reductase, and poly(hydroxyalkanoate) synthase. In a further embodiment of the present invention, the nucleic acid sequences are derived from the following classes of enzymes: ketosynthase-acyltransferases, chain length factors, acyl carrier proteins, and cyclases. The nucleic acid sequences employed in the methods of the present invention can be various forms of deoxyribonucleic acid (for example, genomic DNA, cDNA, sense-strand sequences, antisense-strand sequences, recombinant DNA, shuffled DNA, modified DNA, or DNA analogs). Alternatively, ribonucleic acid (including, but not limited to, genomic RNA, messenger RNA, catalytic RNA, sense-strand sequences, antisense-strand sequences, recombinant RNA, shuffled RNA, modified RNA, or RNA analogs) can be used. Individual nucleic acid sequences or libraries of nucleic acid sequences can be employed in synthesis of the gene fusion construct. The nucleic acid sequences encoding the enzymatic domains can be joined directly to one another, or they can be joined via one or more nucleotide linker sequences ranging in length from about three to about three hundred nucleotides. Optionally, one or more of the nucleic acid sequences, and/or one or more of the linker sequences, can be mutated, shuffled, or otherwise altered (either prior to, or after cojoining of the sequences).

As with the gene fusion constructs and modified gene fusion constructs described above, the nucleic acids incorporated into the gene fusion constructs of the present methods can be derived from a single metabolic pathway, or they can be derived from two or more distinct metabolic pathways (e.g., to produce a novel metabolic pathway). In addition, the nucleic acids incorporated into the gene fusion constructs can be derived from a single source or species, or they can originate from multiple sources or species. The gene fusion constructs and modified gene fusion constructs can comprise a library of constructs, such as recombinant gene fusion constructs, which can optionally be screened prior to introducing the gene fusion construct or modified gene fusion construct into the biological system. In addition, one or more transcription regulatory sequences can be incorporated into the gene fusion construct. The biological system can be a prokaryotic system, for example, a bacterial or archeabacterial cell; alternatively, the biological system can be a eukaryotic system, for example, a eukaryotic cell, a plant cell, an animal cell, a fungus, a yeast, a protoplast, a tissue culture, an organism, and the like. Introduction of the gene fusion construct into any of these systems can be achieved, for example, by techniques known to one in the art, such as electroporation, microinjection, particle bombardment, polyethylene glycol-mediated transformation, or Agrobacterium-mediated transformation. The methods of the present invention can further include the step of expressing the gene fusion construct in the eukaryotic system. Furthermore, the present invention provides gene fusion constructs, vectors comprising the gene fusion constructs, hybrid proteins, and transgenic biological systems, such as transgenic plant systems, as prepared by the methods of the present invention.

In addition, the present invention provides recombinant nucleic acid sequences prepared by the methods described herein. In some embodiments, the recombinant nucleic acid sequences comprise sequences encoding at least two cojoined enzymatic domains derived from different eukaryotic species or from a eukaryote and a prokaryote. In alternative embodiments, the recombinant nucleic acid sequences comprise sequences encoding at least two cojoined enzymatic domains derived from plant genes. Optionally, at the recombinant nucleic acid sequence is modified, for example, by mutation, shuffling, recursive combination, and the like. In some embodiments, the recombinant nucleic acid sequences comprise sequences encoding at least two cojoined enzymatic domains, wherein the sequence encoding one or more of the enzymatic domains has been modified as described herein. The enzymatic domains encoded by the recombinant nucleic acid sequences can be derived from proteins that participate in the same metabolic pathway, or they can be derived from two or more distinct metabolic pathways (e.g., to produce a novel metabolic pathway). Examples of metabolic pathways from which enzymatic domains can be derived include the carotene synthetic pathway (including phytoene synthase, phytoene desaturase, and beta-cyclase), the ectoine synthetic pathway (including diaminobutyric acid aminotransferase, diaminobutyric acid acetyltransferase, and ectoine synthase), the poly(hydroxyalkanoate) synthetic pathway (including a beta ketothiolase, a reductase, and a poly(hydroxyalkanoate) synthase) and a minimal polyketide synthetic pathway (including a ketosynthase-acyltransferase, a chain-length factor, an acyl carrier protein, and a cyclase).

### BRIEF DESCRIPTION OF THE DRAWINGS

FIG. 1: Schematic of modified gene fusion construct having two nucleic acid sequences, without a linker sequence. The stop codon in gene 1 is removed and then fused in frame to the coding sequence in gene 2.

FIG. 2: Schematic of modified gene fusion construct having two nucleic acid sequences, with a linker sequence. The stop codon in gene 1 is removed and then fused with a linker sequence that is fused in frame to the coding sequence in gene 2.

FIG. 3: Schematic of gene fusion construct having three nucleic acid sequences, with and without linker sequences. The presence of linker sequences is optional. The stop codons in genes 1 and 2 are removed prior to in-frame fusion to gene 3.

FIG. 4: Carotenoid biosynthesis pathway, and potential embodiments of the gene fusion products of the present invention.

FIG. 5: Ectoine biosynthesis pathway. (ASA, aspartic β-semialdehyde; DABA, 2,4-diaminobutyric acid; ADABA, γ-N-acetyl-α,γ-diaminobutyric acid)

FIG. 6: PHA biosynthesis pathway. (R=acetyl, propionyl, and other longer chain groups; i and j=numbers of repeated units. The variations of the final polymers are determined by the R-groups from the initial building block.)

FIG. 7: Minimal polyketide synthesis pathway.

FIG. 8: Cloning strategy for functional isolation of the wild type ectoine synthase operon.

FIG. 9: Strategy for making the fusion construct of three ectoine biosynthesis enzymes.

FIG. 10: Growth of E. coli transformed with pBR322 (control) and the plasmid containing WT ect operon (ect operon 1 and ect operon 2 are two individual transformed E. coli colonies) and the plasmid containing fused ect genes (fused ect 1 and fused ect2 are two individual transformed E. coli colonies) at different salt concentrations.

### DETAILED DISCUSSION OF THE INVENTION

Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a device” includes a combination of two or more such devices, reference to “a gene fusion construct” includes mixtures of constructs, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

In describing and claiming the present invention, the following terminology will be used in accordance with the definitions set out below.

The term “modified nucleic acid sequence” refers to a nucleic acid sequence which has been altered as compared to one or more parental nucleic acid(s) (e.g., such as one or more naturally occurring nucleic acid(s)), e.g., by modifying, deleting, rearranging, or replacing one or more nucleotide residue in a modified nucleic acid as compared to the parental nucleic acid. Preferred modes of nucleic acid sequence modification include shuffling and mutation. In some preferred embodiments of the invention, the modification to a nucleic acid sequences results in a substitution, deletion and/or insertion at an internal region of an amino acid sequence encoded by the nucleic acid sequence, and more preferably a plurality of internal modifications (i.e., two, three, or more) are introduced in the encoded polypeptide. This type of internal modification is to be distinguished from, for example, the truncation of one terminus of a protein. It follows that the site of an internal modification to an enzymatic domain is flanked by amino and carboxyl terminals of that enzymatic domain.

The terms “modified protein,” “modified enzyme” and “modified enzymatic domain” refer to translation products encoded by the corresponding modified nucleic acid sequence.

The terms “diversification” and “diversity,” as applied to a nucleic acid sequence, refers to generation of a plurality of modified forms of a parental nucleic acid, or plurality of parental nucleic acids. In the case where the nucleic acid sequence encodes a gene product, diversity in the nucleic acid sequence can result in diversity in the corresponding gene product, e.g., a diverse pool of nucleic acid sequences encoding a plurality of modified proteins. In some preferred embodiments of the invention, this sequence diversity is be exploited by screening/selecting for modified nucleic acids and/or proteins possessing desirable functional attributes.

The term “encoding” refers to a polynucleotide sequence encoding one or more amino acids. The term does not require a start or stop codon. An amino acid sequence can be encoded in any one of six different reading frames provided by a polynucleotide sequence.

The term “plant” includes whole plants, shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

The term “gene fusion construct” as used herein refers to a recombinant nucleic acid sequence comprising cojoined sequences derived from at least two different parental nucleic acids. A “modified gene fusion construct” comprises a subset of gene fusion constructs, in which at least one nucleotide (optionally, in a coding region or linker region) in the construct is modified, or changed, as compared to a parent or wild-type sequence from which that portion of the construct was derived.

The term “enzymatic domain” refers to the portion of an amino acid sequence in a polypeptide or protein that encompasses an active site of the enzyme. The term “active site of an enzyme” generally refers to a region of the enzyme capable of effecting some sort of functional activity of the protein, e.g., catalyze a chemical reaction, bind to a ligand or substrate, or specifically interact with another molecule such as a small molecule, biopolymer, nucleic acid, or other protein or peptide. The activity of the protein can be an activity endogenous to the naturally-occurring form of the protein, or can be an activity that has been introduced into the protein by modification of the parental nucleic acid from which it was derived.

An enzymatic domain is “derived from” a specified enzyme if it corresponds to some portion of the amino acid sequence of that enzyme, or in some cases substantially all of the amino acid sequence of that enzyme. An enzymatic domain is considered derived from a specified enzyme even if it has a substantially different sequence and/or function as the result of modification of the nucleic acid sequence encoding the specified enzyme.

A nucleic acid sequence is “derived from” a plant if the sequence was originally isolated from a plant, regardless of whether the sequence is subsequently modified as described herein.

The terms “peptide linker” and “peptide linker sequence” refer to amino acid sequences that are positioned between other peptide sequences (e.g., enzymatic domains), linking these sequences together. The peptide linkers can act, for example, as spacer units in a final extended construct. Alternatively, the peptide linkers can provide a mechanism by which the linked sequences can be separated (for example, by providing proteolytic cleavage sites or intein sequences).

The term “gene fusion construct” refers to a construct comprising two or more cojoined heterologous nucleic acid sequences. In preferred embodiments of the invention the cojoined sequences encode heterologous enzyme domains, and expression of the construct results in a hybrid protein comprising the heterologous enzyme domains, fused together either directly or through a peptide linker. Preparation of the gene fusion construct typically entails maintaining the correct reading frame in the fused coding regions and removal of any internal stop codons. Alternatively, internal stop codons can be suppressed in certain biological systems.

The term “heterologous” as used herein describes a relationship between two or more components which indicates that the components are not normally found in proximity to one another in nature. Thus, the term “heterologous enzyme domains” refers to enzyme domains which are not found in a single polypeptide in nature, e.g., where the heterologous domains are derived from two different enzymes, or different species of an enzyme, or the like. The heterologous items (i.e., enzyme domains, polypeptides, nucleic acid sequences, and the like) can be derived from the same species (e.g., two different proteins in the species), or from different species.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g., a genetically engineered coding sequence or an allele from a different ecotype or variety).

The term “metabolic pathway” refers to any combination of catalytic activities, typically enzyme-mediated, that result in the chemical conversion of a substrate to a product. A metabolic pathway can be catabolic or anabolic. A metabolic pathway can be one that is normally found in a biological system, or can be a novel metabolic pathway not found in nature. A group of two or more enzymes (or enzymatic domains) are members of a common metabolic pathway if a substrate and/or product of each enzyme is a substrate or product for another member of the group, and the coordinated activities of the enzymes will, under the proper conditions, result in the conversion of a substrate (or substrates) to a product (or products) through an intermediate (or series of intermediates). In a typical example, a substrate is converted into a first intermediate by a first member of the group, the first intermediate is converted into a second intermediate by a second member of the group, and the second intermediate is converted into the final product of the metabolic pathway by a third member of the group. The number of intermediates in a metabolic pathway varies with the pathway, e.g., some pathways have only a single intermediate, others have many. In some cases a metabolic pathway can branch, so that one or more intermediates can be converted into alternative products. Depending upon the metabolic pathway, the number of substrates, products and intermediates can vary from one to many.

The term “biological system” refers to any system in which a nucleic acid sequence can be introduced for subsequent replication, recombination and/or expression, including, but not limited to, bacteria, archaebacteria, protazoa, fungi, plants, animals, viruses, single cells, multicellular organisms, artificial structures such as liposomes, in vitro expression systems, and the like.

Metabolic Pathway Engineering and Expression in Eukaryotes

Establishing a new (or modified) metabolic pathway having multiple single enzymes in a desired compartment of a eukaryote, such as a plant, is more difficult than achieving this in the relatively uncompartmentalized environment of a prokaryotic system. The difficulty lies in part with the fact that transcription of each enzyme is governed by its own promoter and termination sequences. As an example, a metabolic pathway consisting of four enzymes typically requires four promoter sequences and four termination sequences for complete expression. After the separate synthesis and translation of the multiple transcripts, difficulty can arise in colocalization of the translated peptide sequences to the same compartment in the transformed host. Another consideration is the source of the enzymes to be engineered into the eukaryote. While the enzymes may participate in the same metabolic pathway, the optimal choice of enzyme for each step of the metabolic pathway may be derived from different species, and thus have varying pH optimums, temperature requirements, turnover rates, and other environment requirements or effects.

One current approach to solving the problem of coexpression of the multiple metabolic enzymes includes cloning nucleic acid sequences encoding each of the enzymes into separate plasmids. The plasmids are then transfected into the desired eukaryotic system via transformation methodologies appropriate for that system (bacterial-mediated transformation, protoplast fusion techniques, particle bombardment, and the like). Alternatively, the nucleic acid sequences encoding the enzymes can be grouped into an expression cassette and transfected into the host cell as a single vector, rather than multiple elements. Such methodologies are known in the art (see, for example, Current Protocols in Molecular Biology, F. M. Ausubel, et al., eds., (a joint venture between Greene Publishing Associates, Inc. and John Wiley & Sons, Inc., supplemented through 2000)).

However, these approaches suffer from considerable drawbacks. If the nucleic acid sequences are incorporated into the host genome, there can be expression problems due to positional effects (for example, the relevant nucleic acid sequence may have inserted into a tightly packed section of chromatin). Segregation effects, as the genome is replicated and the host cells divide, can also lead to loss of one or more of the relevant sequences. In addition, there are stability issues associated with repeated use of the same promoter systems, in the case of the tandem cloning approach. These problems severely impair the practicality and implementation of multi-enzymatic metabolic pathways in eukaryotic systems.

The present invention provides methods for expressing a plurality of enzymatic activities, and methods of producing modified gene constructs, in which the desired metabolic enzymes are produced as a single, extended, multi-functional hybrid protein. By synthesizing the desired enzymatic domains as a single peptide translated from a series of cojoined nucleic acid sequences, the issues surrounding coexpression and colocalization of the multiple enzymes in the metabolic pathway are overcome. The nucleic acid sequences incorporated into the gene fusion constructs of the present invention can be directly linked to one another, or the sequences can be separated by nucleotide linker sequences. In some embodiments of the present invention, the enzyme activities incorporated into the resulting hybrid protein will be active in this cojoined, or tethered, form. In alternative embodiments, it may be desirable to cleave, or separate the enzymatic domains after transcription or translation in order to, for example, modify the enzymatic activity. Separation of the component enzymatic activities can be accomplished, for example, through the use of peptide linkers that are sensitive to proteolytic cleavage or hydrolysis, or by incorporation of intein or intron sequences into the linker sequences. These methods, and the gene fusion constructs, modified gene fusion constructs, and hybrid proteins employed in or produced by these methods, are described in further detail below.

Gene Fusion Constructs

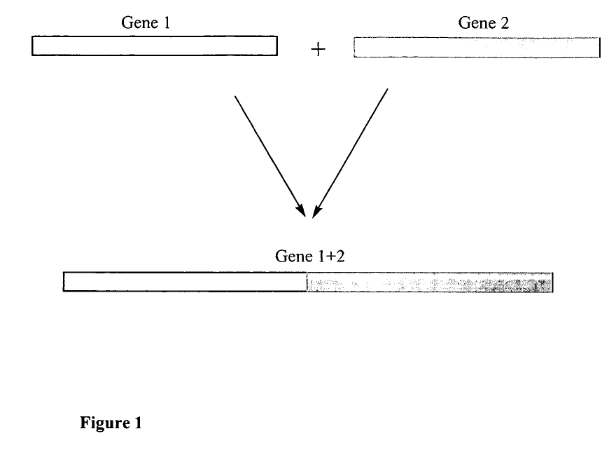

The present invention provides methods for expressing a plurality of enzyme activities through the use of gene fusion constructs, as well as methods for producing modified gene constructs. In addition, the present invention provides the gene fusion constructs for use in these methods, and the modified gene fusion constructs prepared by these methods. Gene fusion constructs in their simplest form are combinations of nucleic acid sequences encoding enzymatic domains (FIGS. 1-3). The constructs can further include nucleic acid sequences that participate in expression of the encoded hybrid protein, such as transcription elements, promoters, termination sequences, introns, and the like. In addition, the constructs can include nucleotide linker sequences such as those described below.

The nucleic acid sequences cojoined to form the gene fusion constructs and modified gene fusion constructs of the present invention can be various forms of deoxyribonucleic acid (for example, genomic DNA, cDNA, sense-strand sequences, antisense-strand sequences, recombinant DNA, shuffled DNA, modified DNA, or DNA analogs). Alternatively, the nucleic acid sequences can be ribonucleic acid (including, but not limited to, genomic RNA, messenger RNA, catalytic RNA, sense-strand sequences, antisense-strand sequences, recombinant RNA, shuffled RNA, modified RNA, or RNA analogs). The nucleic acid sequences incorporated into the fusion constructs of the present invention can also be derived from one or more libraries of nucleic acid sequences.

The gene fusion constructs and modified gene fusion constructs of the present invention can be prepared by a number of techniques known in the art, such as molecular cloning techniques. A wide variety of cloning and in vitro amplification methods suitable for the construction of recombinant nucleic acids, such as expression vectors, are well-known to persons of skill. General texts which describe molecular biological techniques useful herein, including mutagenesis, include Berger and Kimmel, Guide to Molecular Cloning Techniques, Methods in Enzymology, volume 152 Academic Press, Inc., San Diego, Calif. (“Berger”); Sambrook, et al., Molecular Cloning—A Laboratory Manual (2nd Ed.), volumes 1-3, Cold Spring Harbor Laboratory, Cold Spring Harbor, N.Y., 1989 (“Sambrook”); and Current Protocols in Molecular Biology, F. M. Ausubel, et al., eds., Current Protocols, a joint venture between Greene Publishing Associates, Inc. and John Wiley & Sons, Inc., (supplemented through 2000) (“Ausubel”)). Examples of techniques sufficient to direct persons of skill through in vitro amplification methods, including the polymerase chain reaction (PCR) the ligase chain reaction (LCR), Qβ-replicase amplification and other RNA polymerase mediated techniques (e.g., NASBA) are found in Berger, Sambrook, and Ausubel, as well as Mullis, et al., (1987) U.S. Pat. No. 4,683,202; PCR Protocols A Guide to Methods and Applications (Innis, et al., eds.) Academic Press Inc. San Diego, Calif. (1990); Arnheim & Levinson (Oct. 1, 1990) Chemical and Engineering News 36-47; The Journal Of NIH Research (1991) 3:81-94; Kwoh, et al., (1989) Proc Natl Acad. Sci. USA 86:1173; Guatelli, et al., (1990) Proc Natl Acad Sci USA 87:1874; Lomell, et al., (1989) J Clin Chem 35:1826; Landegren, et al., (1988) Science 241:1077-1080; Van Brunt (1990) Biotechnology 8:291-294; Wu and Wallace, (1989) Gene 4:560; Barringer, et al., (1990) Gene 89:117, and Sooknanan and Malek (1995) Biotechnology 13:563-564. Improved methods of cloning in vitro amplified nucleic acids are described in Wallace, et al., U.S. Pat. No. 5,426,039. Improved methods of amplifying large nucleic acids by PCR are summarized in Cheng, et al., (1994) Nature 369:684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, Ausbel, Sambrook and Berger, all supra.

The isolation of a nucleic acid sequence for inclusion in a gene fusion construct may be accomplished by any number of techniques known in the art. For instance, oligonucleotide probes based on known sequences can be used to identify the desired gene in a cDNA or genomic DNA library. Probes may be used to hybridize with genomic DNA or cDNA sequences to isolate homologous genes in the same or different species. Alternatively, antibodies raised against an enzyme can be used to screen an mRNA expression library for the corresponding coding sequence.

Alternatively, the nucleic acids of interest can be amplified from nucleic acid samples using amplification techniques. For instance, polymerase chain reaction (PCR) technology can be used to amplify the sequences of desired gene directly from genomic DNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. For a general overview of PCR, see PCR Protocols: A Guide to Methods and Applications. (Innis, M, Gelfand, D., Sninsky, J. and White, T., eds.), Academic Press, San Diego (1990).

Polynucleotides may also be synthesized by well-known techniques as described in the technical literature. See, e.g., Carruthers, et al., Cold Spring Harbor Symp Quant Biol 47:411-418 (1982), and Adams, et al., J Am Chem Soc 105:661 (1983). Double stranded DNA fragments may then be obtained either by synthesizing the complementary strand and annealing the strands together under appropriate conditions, or by adding the complementary strand using DNA polymerase with an appropriate primer sequence.

Oligonucleotides for use as probes, e.g., in in vitro amplification methods, for use as gene probes, or as shuffling targets (e.g., synthetic genes or gene segments) are typically synthesized chemically according to the solid phase phosphoramidite triester method described by Beaucage and Caruthers (1981) Tetrahedron Letts 22(20):1859-1862, e.g., using an automated synthesizer, as described in Needham-VanDevanter, et al., (1984) Nucleic Acids Res 12:6159-6168. Oligonucleotides for use in the nucleic acid constructs of the present invention can also be custom made and ordered from a variety of commercial sources known to persons of skill.

In some embodiments of the present invention, the gene fusion constructs include elements in addition to the cojoined nucleic acid sequences, such as promoters, enhancer elements, and signaling sequences. Exemplary promoters include the CaMV promoter, a promoter from the ribulose-1,5-bisphosphate carboxylase-oxygenase small subunit gene, a ubiquitin promoter, and a rolD promoter. Exemplary enhancer elements include, but are not limited to, Exemplary signaling sequences include, but are not limited to, nucleic acid sequences encoding tissue-specific transit peptides (for example, a chloroplast transit peptide).

In some embodiments of the present invention, gene fusion constructs and/or modified gene fusion constructs suitable for transformation of plant cells are prepared. A DNA sequence coding for the desired nucleic acid, for example a cDNA or a genomic sequence encoding an enzymatic domain, is conveniently used to construct a recombinant expression cassette which can be introduced into the desired plant. An expression cassette will typically comprise a selected nucleic acid sequence (modified or unmodified, depending upon the construct) operably linked to a promoter sequence and other transcriptional and translational initiation regulatory sequences which will direct the transcription of the sequence from the gene in the intended tissues (e.g., entire plant, leaves, seeds) of the transformed plant.

For example, a strongly or weakly constitutive plant promoter can be employed which will direct expression of the encoded sequences in a gene fusion construct or modified gene fusion construct as set forth herein in all tissues of a plant. Such promoters are active under most environmental conditions and states of development or cell differentiation. Examples of constitutive promoters include the 1′- or 2′-promoter derived from T-DNA of Agrobacterium tumefaciens, and other transcription initiation regions from various plant genes known to those of skill. In situations in which overexpression of a gene from a gene fusion construct is detrimental to the plant, one of skill, upon review of this disclosure, will recognize that weak constitutive promoters can be used for low-levels of expression. In those cases where high levels of expression is not harmful to the plant, a strong promoter, e.g., a t-RNA or other pol III promoter, or a strong pol II promoter, such as the cauliflower mosaic virus promoter, can be used.

Alternatively, a plant promoter may be under environmental control. Such promoters are referred to here as “inducible” promoters. Examples of environmental conditions that may effect transcription by inducible promoters include pathogen attack, anaerobic conditions, or the presence of light.

The promoters incorporated into the gene fusion constructs and/or modified gene fusion constructs of the present invention can be “tissue-specific” and, as such, under developmental control in that the desired gene is expressed only in certain tissues, such as leaves and seeds. In embodiments in which one or more nucleic acid sequences endogenous to the plant system are incorporated into the construct, the endogenous promoters (or variants thereof) from these genes can be employed for directing expression of the genes in the transfected plant. Tissue-specific promoters can also be used to direct expression of heterologous structural genes, including modified nucleic acids as described herein.

In general, the particular promoter used in the expression cassette in plants depends on the intended application. Any of a number of promoters which direct transcription in plant cells is suitable. The promoter can be either constitutive or inducible. In addition to the promoters noted above, promoters of bacterial origin which operate in plants include the octopine synthase promoter, the nopaline synthase promoter and other promoters derived from native Ti plasmids (see, Herrara-Estrella, et al., (1983) Nature 303:209-213). Viral promoters include the 35S and 19S RNA promoters of cauliflower mosaic virus (Odell, et al., (1985) Nature 313:810-812). Other plant promoters include the ribulose-1,3-bisphosphate carboxylase small subunit promoter and the phaseolin promoter. The promoter sequence from the E8 gene and other genes may also be used. The isolation and sequence of the E8 promoter is described in detail in Deikman and Fischer (1988) EMBO J. 7:3315-3327.

To identify candidate promoters, the 5′ portions of a genomic clone is analyzed for sequences characteristic of promoter sequences. For instance, promoter sequence elements include the TATA box consensus sequence (TATAAT), which is usually 20 to 30 base pairs upstream of the transcription start site. In plants, further upstream from the TATA box, at positions −80 to −100, there is typically a promoter element with a series of adenines surrounding the trinucleotide G (or T) as described by Messing, et al., (1983) Genetic Engineering in Plants, Kosage, et al., (eds.), pp. 221-227.

In preparing gene fusion constructs or modified gene fusion constructs of the invention, sequences other than the promoter and the cojoined nucleic acid sequences can also be employed. If normal polypeptide expression is desired, a polyadenylation region at the 3′-end of the shuffled coding region can be included. The polyadenylation region can be derived from the natural gene, from a variety of other plant genes, or from T-DNA.

The gene fusion construct and/or modified gene fusion construct can also include a marker gene which confers a selectable phenotype on plant cells. For example, the marker may encode biocide tolerance, particularly antibiotic tolerance, such as tolerance to kanamycin, G418, bleomycin, hygromycin, or herbicide tolerance, such as tolerance to chlorosluforon, or phosphinothricin (the active ingredient in the herbicides bialaphos and Basta).

The gene fusion construct may also comprise a coding sequence or fragment thereof fused in-frame to a marker sequence which, e.g., facilitates purification of the encoded polypeptide. Such purification facilitating domains include, but are not limited to, metal chelating peptides such as histidine-tryptophan modules that allow purification on immobilized metals, a sequence which binds glutathione (e.g., GST), a hemagglutinin (HA) tag (corresponding to an epitope derived from the influenza hemagglutinin protein; Wilson, I., et al., (1984) Cell 37:767), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the enzymatic domains is useful to facilitate purification.

For example, one expression vector possible to use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography, as described in Porath, et al., (1992) Protein Expression and Purification 3:263-281) while the enterokinase cleavage site provides a method for separating the polyhistidine region from the rest of the expression product. pGEX vectors (Amersham Pharmacia Biotech) are optionally used to express foreign polypeptides as fusion proteins with glutathione S-transferase (GST). Other expression systems, such as, e.g., pPICz vectors (Invitrogen) that allow for expression in Pichia are also optionally used. In general, such fusion proteins are soluble and can easily be purified from lysed cells by adsorption to ligand-agarose beads (e.g., glutathione-agarose in the case of GST-fusions) followed by elution in the presence of free ligand.

Polypeptides of the invention can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. In some cases the protein will need to be refolded to recover a functional product. In addition to the references noted supra, a variety of purification methods are well known in the art, including, e.g., those set forth in Sandana (1997) Bioseparation of Proteins, Academic Press, Inc.; and Bollag, et al., (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

Cell-free transcription/translation systems can also be employed to express a gene fusion construct of the present invention. Several such systems are commercially available. A general guide to in vitro transcription and translation protocols is found in Tymms (1995) In vitro Transcription and Translation Protocols: Methods in Molecular Biology Volume 37, Garland Publishing, NY.

The invention also includes compositions comprising two or more nucleic acids of the invention (e.g., as substrates for recombination). The composition can comprise a library of recombinant nucleic acids, where the library contains at least 2, at least 3, at least 5, at least 10, at least 20 or at least 50 or more nucleic acids. The nucleic acids are optionally cloned into expression vectors, providing expression libraries.

The invention also includes compositions produced by digesting one or more nucleic acids of the invention with a restriction endonuclease, an RNAse, or a DNAse (e.g., as is performed in certain of the recombination formats noted above); and compositions produced by fragmenting or shearing one or more polynucleotide of the invention by mechanical means (e.g., sonication, vortexing, and the like), which can also be used to provide substrates for recombination in the methods above. Similarly, compositions comprising sets of oligonucleotides corresponding to more than one nucleic acid of the invention are useful as recombination substrates and are a feature of the invention. For convenience, these fragmented, sheared, or oligonucleotide synthesized mixtures are referred to as fragmented nucleic acid sets.

Also included in the invention are compositions produced by incubating one or more of the fragmented nucleic acid sets in the presence of ribonucleotide or deoxyribonucleotide triphosphates and a nucleic acid polymerase. This resulting composition forms a recombination mixture for many of the recombination formats noted above. The nucleic acid polymerase may be an RNA polymerase, a DNA polymerase, or an RNA-directed DNA polymerase (e.g., a “reverse transcriptase”); the polymerase can be, e.g., a thermostable DNA polymerase (such as, VENT, TAQ, or the like).

Recombinant methods for producing and isolating fusion proteins of the invention are described above. In addition to recombinant production, the polypeptides may be produced by direct peptide synthesis using solid-phase techniques (see, e.g., Stewart, et al., (1969) Solid-Phase Peptide Synthesis, WH Freeman Co, San Francisco; Merrifield J (1963) J Am Chem Soc 85:2149-2154). Peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. For example, subsequences may be chemically synthesized separately and combined using chemical methods to provide full-length fusion proteins. Alternately, such sequences may be ordered from any number of companies which specialize in production of polypeptides. Most commonly fusion proteins of the invention are produced by expressing coding nucleic acids and recovering polypeptides, e.g., as described above.

Modification of Nucleic Acid Sequences to Form Modified Gene Fusion Constructs

In some embodiments of the present invention, modified gene fusion constructs are employed. The process of modifying one or more of the nucleic acid sequences in the gene fusion construct comprises altering the sequence, as compared to the originally-identified or “parental” sequence for that protein or enzymatic domain. The process of altering the sequence can result in, for example, single nucleotide substitutions, multiple nucleotide substitutions, and insertion or deletion of regions of the nucleic acid sequence.

A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the alteration, engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids that encode proteins with or which confer desirable properties. Following diversification by one or more of the methods herein, or otherwise available to one of skill, any nucleic acids that are produced can be selected for a desired activity or property, e.g., the encoding of multiple enzymatic domains derived from one or more metabolic pathways. This can include identifying any activity or set of activities that can be detected, for example, in an automated or automatable format, by any of the assays in the art. For example, the biosynthesis of carotenoid compounds, ectoine, various polyhydroxyalkanoates, numerous aromatic polyketides, or other metabolic pathway products or byproducts can be determined, as described further below. Alternatively, individual enzymatic activities can be assayed by any of a number of assays known in the art. In addition, a variety of related (or even unrelated) properties can be evaluated, in serial or in parallel, at the discretion of the practitioner.

Descriptions of a variety of diversity generating procedures for generating modified nucleic acid sequences encoding multiple enzymatic domains are found the following publications and the references cited therein: Soong, N., et al., (2000) “Molecular breeding of viruses” Nat Genet 25(4):436-39; Stemmer, et al., (1999) “Molecular breeding of viruses for targeting and other clinical properties” Tumor Targeting 4:1-4; Ness, et al., (1999) “DNA Shuffling of subgenomic sequences of subtilisin” Nature Biotechnology 17:893-896; Chang, et al., (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians, et al., (1999) “Directed evolution of thymidine kinase for AZT phosphorylation using DNA family shuffling” Nature Biotechnology 17:259-264; Crameri, et al., (1998) “DNA shuffling of a family of genes from diverse species accelerates directed evolution” Nature 391:288-291; Crameri, et al., (1997) “Molecular evolution of an arsenate detoxification pathway by DNA shuffling,” Nature Biotechnology 15:436-438; Zhang, et al., (1997) “Directed evolution of an effective fucosidase from a galactosidase by DNA shuffling and screening” Proc Natl Acad Sci USA 94:4504-4509; Patten, et al., (1997) “Applications of DNA Shuffling to Pharmaceuticals and Vaccines” Current Opinion in Biotechnology 8:724-733; Crameri, et al., (1996) “Construction and evolution of antibody-phage libraries by DNA shuffling” Nature Medicine 2:100-103; Crameri, et al., (1996) “Improved green fluorescent protein by molecular evolution using DNA shuffling” Nature Biotechnology 14:315-319; Gates, et al., (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology VCH Publishers, New York. pp. 447-457; Crameri and Stemmer (1995) “Combinatorial multiple cassette mutagenesis creates all the permutations of mutant and wildtype cassettes” Bio Techniques 18:194-195; Stemmer, et al., (1995) “Single-step assembly of a gene and entire plasmid form large numbers of oligodeoxy-ribonucleotides” Gene 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc Natl Acad Sci USA 91:10747-10751.

Mutational methods of generating diversity include, for example, site-directed mutagenesis (Ling, et al., (1997) “Approaches to DNA mutagenesis: an overview” Anal Biochem 254(2):157-178; Dale, et al., (1996) “Oligonucleotide-directed random mutagenesis using the phosphorothioate method” Methods Mol Biol 57:369-374; Smith (1985) “In vitro mutagenesis” Ann. Rev Genet 19:423-462; Botstein & Shortle (1985) “Strategies and applications of in vitro mutagenesis” Science 229:1193-1201; Carter (1986) “Site-directed mutagenesis” Biochem J 237:1-7; and Kunkel (1987) “The efficiency of oligonucleotide directed mutagenesis” in Nucleic Acids & Molecular Biology (Eckstein, F. and Lilley, D. M. J., eds., Springer Verlag, Berlin)); mutagenesis using uracil containing templates (Kunkel (1985) “Rapid and efficient site-specific mutagenesis without phenotypic selection” Proc Natl Acad Sci USA 82:488-492; Kunkel, et al., (1987) “Rapid and efficient site-specific mutagenesis without phenotypic selection” Methods in Enzymol 154:367-382; and Bass, et al., (1988) “Mutant Trp repressors with new DNA-binding specificities” Science 242:240-245); oligonucleotide-directed mutagenesis (Methods in Enzymol 100:468-500 (1983); Methods in Enzymol 154:329-350 (1987); Zoller & Smith (1982) “Oligonucleotide-directed mutagenesis using M13-derived vectors: an efficient and general procedure for the production of point mutations in any DNA fragment” Nucleic Acids Res 10:6487-6500; Zoller & Smith (1983) “Oligonucleotide-directed mutagenesis of DNA fragments cloned into M13 vectors” Methods in Enzymol 100:468-500; and Zoller & Smith (1987) “Oligonucleotide-directed mutagenesis: a simple method using two oligonucleotide primers and a single-stranded DNA template” Methods in Enzymol 154:329-350); phosphorothioate-modified DNA mutagenesis (Taylor, et al., (1985) “The use of phosphorothioate-modified DNA in restriction enzyme reactions to prepare nicked DNA” Nucl Acids Res 13:8749-8764; Taylor, et al., (1985) “The rapid generation of oligonucleotide-directed mutations at high frequency using phosphorothioate-modified DNA” Nucl Acids Res 13:8765-8787 (1985); Nakamaye & Eckstein (1986) “Inhibition of restriction endonuclease Nci I cleavage by phosphorothioate groups and its application to oligonucleotide-directed mutagenesis” Nucl Acids Res 14:9679-9698; Sayers, et al., (1988) “Y-T Exonucleases in phosphorothioate-based oligonucleotide-directed mutagenesis” Nucl Acids Res 16:791-802; and Sayers, et al., (1988) “Strand specific cleavage of phosphorothioate-containing DNA by reaction with restriction endonucleases in the presence of ethidium bromide” Nucl Acids Res 16:803-814); mutagenesis using gapped duplex DNA (Kramer, et al., (1984) “The gapped duplex DNA approach to oligonucleotide-directed mutation construction” Nucl Acids Res 12:9441-9456; Kramer and Fritz (1987) “Oligonucleotide-directed construction of mutations via gapped duplex DNA” Methods in Enzymol 154:350-367; Kramer, et al., (1988) “Improved enzymatic in vitro reactions in the gapped duplex DNA approach to oligonucleotide-directed construction of mutations” Nucl Acids Res 16:7207; and Fritz, et al., (1988) “Oligonucleotide-directed construction of mutations: a gapped duplex DNA procedure without enzymatic reactions in vitro” Nucl Acids Res 16:6987-6999).

Additional suitable methods include point mismatch repair (Kramer, et al., (1984) “Point Mismatch Repair” Cell 38:879-887), mutagenesis using repair-deficient host strains (Carter, et al., (1985) “Improved oligonucleotide site-directed mutagenesis using M13 vectors” Nucl Acids Res 13:4431-4443; and Carter (1987) “Improved oligonucleotide-directed mutagenesis using M13 vectors” Methods in Enzymol 154:382-403), deletion mutagenesis (Eghtedarzadeh and Henikoff (1986) “Use of oligonucleotides to generate large deletions” Nucl Acids Res 14: 5115), restriction-selection and restriction-selection and restriction-purification (Wells, et al., (1986) “Importance of hydrogen-bond formation in stabilizing the transition state of subtilisin” Phil Trans R Soc Lond A 317:415-423), mutagenesis by total gene synthesis (Nambiar, et al., (1984) “Total synthesis and cloning of a gene coding for the ribonuclease S protein” Science 223:1299-1301; Sakamar and Khorana (1988) “Total synthesis and expression of a gene for the a-subunit of bovine rod outer segment guanine nucleotide-binding protein (transducin)” Nucl Acids Res 14:6361-6372; Wells, et al., (1985) “Cassette mutagenesis: an efficient method for generation of multiple mutations at defined sites” Gene 34:315-323; and Grundström, et al., (1985) “Oligonucleotide-directed mutagenesis by microscale ‘shot-gun’ gene synthesis” Nucl Acids Res 13:3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc Natl Acad Sci USA 83:7177-7181). Additional details on many of the above methods can be found in Methods in Enzymology Volume 154, which also describes useful controls for trouble-shooting problems with various mutagenesis methods.

Additional details regarding various diversity generating methods can be found in the following US patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer, et al., (Sep. 22, 1998) “Methods for Generating Polynucleotides having Desired Characteristics by Iterative Selection and Recombination;” U.S. Pat. No. 5,830,721 to Stemmer, et al., (Nov. 3, 1998), “DNA Mutagenesis by Random Fragmentation and Reassembly;” U.S. Pat. No. 5,834,252 to Stemmer, et al., (Nov. 10, 1998) “End-Complementary Polymerase Reaction;” U.S. Pat. No. 5,837,458 to Minshull, et al., (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen, et al., “Targeting of Genetic Vaccine Vectors;” WO 99/41383 by Punnonen, et al., “Antigen Library Immunization;” WO 99/41369 by Punnonen, et al., “Genetic Vaccine Vector Engineering;” WO 99/41368 by Punnonen, et al., “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer, et al., “Modification of Virus Tropism and Host Range by Viral Genome Shuffling;” WO 99/21979 by Apt, et al., “Human Papillomavirus Vectors;” WO 98/31837 by del Cardayre, et al., “Evolution of Whole Cells and Organisms by Recursive Sequence Recombination;” WO 98/27230 by Patten and Stemmer, “Methods and Compositions for Polypeptide Engineering;” WO 98/13487 by Stemmer, et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold, et al., “Recombination of Polynucleotide Sequences Using Random or Defined Primers,” WO 99/29902 by Arnold, et al., “Method for Creating Polynucleotide and Polypeptide Sequences,” WO 98/41653 by Vind, “An in Vitro Method for Construction of a DNA Library,” WO 98/41622 by Borchert, et al., “Method for Constructing a Library Using DNA Shuffling,” and WO 98/42727 by Pati and Zarling, “Sequence Alterations using Homologous Recombination,” WO 00/18906 by Patten, et al., “Shuffling of Codon-Altered Genes;” WO 00/04190 by del Cardayre, et al., “Evolution of Whole Cells and Organisms by Recursive Recombination;” WO 00/42561 by Crameri, et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov, et al., “Methods for Making Character Strings, Polynucleotides & Polypeptides Having Desired Characteristics;” WO 01/23401 by Welch, et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Affholter.

Certain U.S. applications provide additional details regarding various diversity generating methods, including “Shuffling of Codon Altered Genes” by Patten, et al., filed Sep. 28, 1999, (U.S. Ser. No. 09/407,800); “Evolution of Whole Cells and Organisms by Recursive Sequence Recombination”, by del Cardayre, et al., filed Jul. 15, 1998 (U.S. Ser. No. 09/166,188), and Jul. 15, 1999 (U.S. Ser. No. 09/354,922); “Oligonucleotide Mediated Nucleic Acid Recombination” by Crameri, et al., filed Sep. 28, 1999 (U.S. Ser. No. 09/408,392), and “Oligonucleotide Mediated Nucleic Acid Recombination” by Crameri, et al., filed Jan. 18, 2000 (PCT/US00/01203); “Use of Codon-Based Oligonucleotide Synthesis for Synthetic Shuffling” by Welch, et al., filed Sep. 28, 1999 (U.S. Ser. No. 09/408,393); “Methods for Making Character Strings, Polynucleotides & Polypeptides Having Desired Characteristics” by Selifonov, et al., filed Jan. 18, 2000, (PCT/US00/01202) and, e.g., “Methods for Making Character Strings, Polynucleotides & Polypeptides Having Desired Characteristics” by Selifonov, et al., filed Jul. 18, 2000 (U.S. Ser. No. 09/618,579); “Methods of Populating Data Structures for Use in Evolutionary Simulations” by Selifonov and Stemmer (PCT/US00/01138), filed Jan. 18, 2000; and “Single-Stranded Nucleic Acid Template-Mediated Recombination and Nucleic Acid Fragment Isolation” by Affholter (U.S. Ser. No. 60/186,482, filed Mar. 2, 2000).

In brief, several different general classes of sequence modification methods, such as mutation, recombination, etc. are applicable to the present invention and set forth, e.g., in the references above. That is, alterations to the component nucleic acid sequences to produced modified gene fusion constructs can be performed by any number of the protocols described, either before cojoining of the sequences, or after the cojoining step. The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc Natl Acad Sci USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre, et al., “Evolution of Whole Cells and Organisms by Recursive Sequence Recombination;” and in, e.g., PCT/US99/15972 by del Cardayre, et al., also entitled “Evolution of Whole Cells and Organisms by Recursive Sequence Recombination.” Thus, any of these processes and techniques for recombination, recursive recombination, and whole genome recombination, alone or in combination, can be used to generate the modified nucleic acid sequences and/or modified gene fusion constructs of the present invention.

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561 by Crameri, et al., “Oligonucleotide Mediated Nucleic Acid Recombination;” WO 01/23401 by Welch, et al., “Use of Codon-Varied Oligonucleotide Synthesis for Synthetic Shuffling;” WO 00/42560 by Selifonov, et al., “Methods for Making Character Strings, Polynucleotides and Polypeptides Having Desired Characteristics;” and WO 00/42559 by Selifonov and Stemmer “Methods of Populating Data Structures for Use in Evolutionary Simulations.”

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 by Selifonov, et al., “Methods for Making Character Strings, Polynucleotides and Polypeptides Having Desired Characteristics” and WO 00/42559 by Selifonov and Stemmer “Methods of Populating Data Structures for Use in Evolutionary Simulations.” Extensive details regarding in silico recombination methods are found in these applications. This methodology is generally applicable to the present invention in providing for recombination of nucleic acid sequences and/or gene fusion constructs encoding proteins involved in various metabolic pathways (such as, for example, carotenoid biosynthetic pathways, ectoine biosynthetic pathways, polyhydroxyalkanoate biosynthetic pathways, aromatic polyketide biosynthetic pathways, and the like) in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see, e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier, et al., (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier, et al., (1999) “Combinatorial Protein Engineering by Incremental Truncation,” Proc Natl Acad Sci USA 96:3562-67; Ostermeier, et al., (1999), “Incremental Truncation as a Strategy in the Engineering of Novel Biocatalysts,” Biological and Medicinal Chemistry 7:2139-44.

Mutational methods which result in the alteration of individual nucleotides or groups of contiguous or non-contiguous nucleotides can be favorably employed to introduce nucleotide diversity into the nucleic acid sequences and/or gene fusion constructs of the present invention. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung, et al., (1989) Technique 1:11-15 and Caldwell, et al., (1992) PCR Methods Applic 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson, et al., (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin and Youvan (1992) Proc Natl Acad Sci USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave and Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g., a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson, et al., (1998) U.S. Pat. No. 5,783,431 “Methods for Generating and Screening Novel Metabolic Pathways,” and Thompson, et al., (1998) U.S. Pat. No. 5,824,485 Methods for Generating and Screening Novel Metabolic Pathways) and their use to identify protein activities of interest has been proposed (In addition to the references noted above, see, Short (1999) U.S. Pat. No. 5,958,672 “Protein Activity Screening of Clones Having DNA from Uncultivated Microorganism”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt, et al., (1998) “Exploiting sequence space: shuffling in vivo formed complementarity determining regions into a master framework” Gene 215:471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “Production of Enzymes Having Desired Activities by Mutagenesis.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson, et al., (1991) “Random mutagenesis of protein sequences using oligonucleotide cassettes” Methods Enzymol 208:564-86; Lim and Sauer (1991) “The role of internal packing interactions in determining the structure and stability of a protein” J Mol Biol 219:359-76; Breyer and Sauer (1989) “Mutational analysis of the fine specificity of binding of monoclonal antibody 51F to lambda repressor” J Biol Chem 264:13355-60); and “Walk-Through Mutagenesis” (Crea, R; U.S. Pat. Nos. 5,830,650 and 5,798,208, and EP Patent 0527809 B1.)

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the present invention can be recombined (with each other, or with related (or even unrelated) sequences) to produce a diverse set of recombinant nucleic acids for use in the gene fusion constructs and modified gene fusion constructs of the present invention, including, e.g., sets of homologous nucleic acids, as well as corresponding polypeptides.

Many of the above-described methodologies for generating modified nucleic acid sequences generate a large number of diverse variants of a parental sequence or sequences. In some preferred embodiments of the invention the modification technique (e.g., some form of shuffling) is used to generate a library of variants that is then screened for a modified nucleic acid or pool of modified nucleic acids encoding some desired functional attribute. This desired functional attribute is preferably an enzymatic activity that is in some way superior to the enzymatic activity encoded by parental sequences. Exemplary enzymatic activities that can be screened for include catalytic rates (conventionally characterized in terms of kinetic constants such as k_{cat} and K_{M}), substrate specificity, and susceptibility to activation or inhibition by substrate, product or other molecules (e.g., inhibitors or activators).

In some preferred embodiments of the invention modified nucleic acids are screened and/or selected by assaying the function of a metabolic pathway in which the expression products of the modified nucleic acids are expected to participate. If the particular modification of a given nucleic acid results in altered function of the gene product, this will often result in a detectable alteration in the output of the pathway. For example, a modification that enhances the activity of an enzymatic domain catalyzes a rate-limiting or partially rate-limiting step in a metabolic pathway will likely increase the rate of product formation in a cell expressing the modified nucleic acid. Thus, modified nucleic acids encoding enhanced enzymatic activities can be identified by screening for host cells producing relatively high levels of the product of the metabolic pathway. One non-limiting example would be a screen for an enhanced activity of an enzyme in a carotenoid synthesis pathway by assaying host cells for increased production of carotenoid. In this example the screening process is facilitated by the color properties of carotenoids, which allows for the detection of improved modified nucleic acids by assaying for increased intensity of visible color associated with the carotenoid.

One example of selection for a desired enzymatic activity entails growing host cells under conditions that inhibit the growth and/or survival of cells that do not sufficiently express an enzymatic activity and/or metabolic pathway of interest. Using such a selection process can eliminate from consideration all modified nucleic acids except those encoding a desired enzymatic activity. For example, in some embodiments of the invention host cells are maintained under conditions that inhibit cell survival in the absence of sufficient levels of the product of an enzyme and/or metabolic pathway of interest. Under these conditions, only a host cell harboring a modified nucleic acid that encodes enzymatic activity or activities able to catalyze production of sufficient levels of the product will survive and grow. For example, a screen for enhanced ectoine synthesis activity can be screened by growing host cells under high salt conditions, as described below in Example 1.

For convenience and high throughput it will often be desirable to screen/select for desired modified nucleic acids in a microorganism, e.g., a bacteria such as E. coli. On the other hand, screening in plant cells or plants can will in some cases be preferable where the ultimate aim is to generate a modified nucleic acid for expression in a plant system.

In some preferred embodiments of the invention throughput is increased by screening pools of host cells expressing different modified nucleic acids, either alone or as part of a gene fusion construct. Any pools showing significant activity can be deconvoluted to identify single clones expressing the desirable activity.

The skilled artisan will recognize that the relevant assay, screening or selection method will vary depending upon the particular enzyme or metabolic pathway. It is normally advantageous to employ an assay that can be practiced in a high-throughput format.

In high through put assays, it is possible to screen up to several thousand different variants in a single day. For example, each well of a microtiter plate can be used to run a separate assay, or, if concentration or incubation time effects are to be observed, every 5-10 wells can test a single variant.

In addition to fluidic approaches, it is possible, as mentioned above, simply to grow cells on media plates that select for the desired enzymatic or metabolic function. This approach offers a simple and high-throughput screening method.

A number of well known robotic systems have also been developed for solution phase chemistries useful in assay systems. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a scientist. Any of the above devices are suitable for application to the present invention. The nature and implementation of modifications to these devices (if any) so that they can operate as discussed herein with reference to the integrated system will be apparent to persons skilled in the relevant art.

High throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization.

The manufacturers of such systems provide detailed protocols for the various high throughput devices. Thus, for example, Zymark Corp. provides technical bulletins describing screening systems for detecting the modulation of gene transcription, ligand binding, and the like. Microfluidic approaches to reagent manipulation have also been developed, e.g., by Caliper Technologies (Mountain View, Calif.).

Optical images viewed (and, optionally, recorded) by a camera or other recording device (e.g., a photodiode and data storage device) are optionally further processed in any of the embodiments herein, e.g., by digitizing the image and/or storing and analyzing the image on a computer. A variety of commercially available peripheral equipment and software is available for digitizing, storing and analyzing a digitized video or digitized optical image, e.g., using PC (Intel x86 or pentium chip compatible DOS™, OS™ WINDOWS™, WINDOWS NT™ or WINDOWS 95™ based machines), MACINTOSH™, or UNIX based (e.g., SUN™ work station) computers.

One conventional system carries light from the assay device to a cooled charge-coupled device (CCD) camera, a common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g. by fluorescent or dark field microscopic techniques.

Target-Activated Non-Functional Fusion of Enzymatic Domains

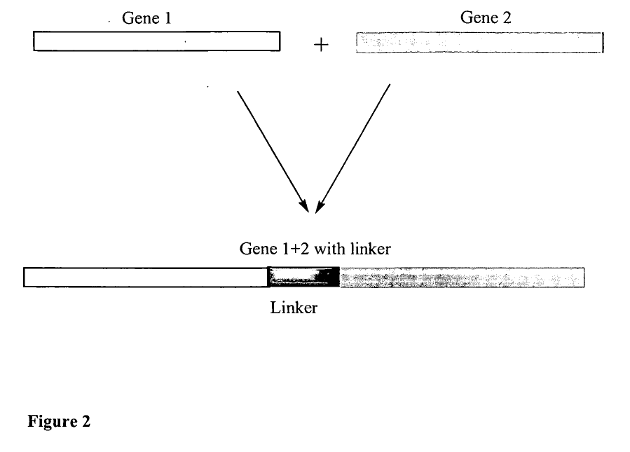

The unmodified and modified nucleic acid sequences employed in the methods of the present invention can be cojoined in a number of manners. For example, the sequences can be joined directly to one another, without any intervening sequences (FIG. 1). Optionally, the stop codon of the first nucleic acid sequence is removed prior to attachment, in frame, to the second nucleic acid sequence. The peptide sequence synthesized based upon such a cojoined sequence would contain the protein sequences (or some portion thereof) attached directly to one another (i.e., the C-terminal amino acid of the first enzymatic domain would be connected to N-terminal of the following enzymatic domain, and so forth). Alternatively, the nucleic acid sequences can be cojoined via one or more nucleotide linker sequences (FIG. 2).

The optional nucleotide linker sequences preferably range in length from about three nucleotides (i.e., encoding a single amino acid linker) to about three hundred nucleotides (i.e., encoding an approximately 100-amino acid linker peptide), but can be longer. Optionally, the nucleotide linker sequences comprise about 12 to about 150 nucleotides, about 12 to about 120 nucleotides or about 12 to about 90 nucleotides. Alternatively, the nucleotide linker sequences comprise about 3 to about 150 nucleotides or about 3 to about 30 nucleotides. The nucleotide linker sequence can be an intron sequence that is removed from the hybrid protein transcript prior to translation. Alternatively, the nucleotide linker sequence can encode a peptide that is translated with the enzymatic domains, as part of the hybrid protein. The peptide encoded by the nucleotide linker sequence can be a random amino acid sequence of any desired composition. One exemplary composition is a peptide linker containing primarily glycines and/or alanines. Another composition option is a peptide linker having an intein structure, such that the peptide linker can extricate itself from the hybrid protein sequence either during or after translation. Preferably, if the nucleotide linker sequence encoding the peptide linker is to be translated as part of the hybrid protein, the length of the linker sequence is in increments of three nucleotides, such that translation of the enzymatic domain encoded after the nucleotide linker sequence is not shifted out of the reading frame.

The linker sequences can also be engineered to contain cleavable sites (such as, for example, a restriction site in the nucleotide linker sequence, or, for example, a protease-susceptible site in the amino acid sequence of the peptide linker).

Incorporation of one or more nucleotide linker sequences into the gene fusion constructs of the present invention provides for further manipulation and control of the gene fusion construct and the resulting hybrid protein products. For example, nucleotide linker sequences can be selected to provide for targeted activation of the hybrid proteins. In such an example of a target-activated hybrid protein, one or more of the enzymatic domains is not activated until the peptide linker region has been modified (for example, cleaved or removed). In an alternative example, the nucleotide linker sequence may affect or inhibit the transcription or translation of the gene fusion construct, unless the nucleotide linker sequence is altered, for example, by cleavage via a catalytic RNA molecule.

Methods for Producing Modified Gene Fusion Constructs

The present invention provides methods for producing a modified gene fusion construct. These methods include the step of cojoining two or more nucleic acid sequences that encode two or more enzymatic domains, where at least one of the nucleic acid sequences has been modified as compared to an originally-determined sequence (FIG. 1). The nucleic acid sequences should be cojoined in a manner such that the reading frame of any downstream coding sequence is maintained. Furthermore, the design should be such that translation of the coding transcript is not prematurely disrupted by a stop codon; this is conveniently achieved by eliminating any internal stop codon from the coding sequence of the construct.

The nucleic acid sequences can be various forms of deoxyribonucleic acid or ribonucleic acid, as described above. In addition, the nucleic acid sequences can optionally comprise individual nucleic acid sequences, or libraries of sequences. Modification to at least one of the nucleic acid sequences can be performed prior to cojoining the two or more sequences together, or it may be achieved after the sequences are cojoined. Such modifications include, but are not limited to, mutation or shuffling of a portion of the nucleic acid sequence.

A gene fusion construct of the invention can optionally be engineered to encode a secretion/localization sequence (e.g., a signal sequence, an organelle targeting sequence, a membrane localization sequence, and the like) and/or a sequence that facilitates purification, e.g., an epitope tag (such as, a FLAG epitope), a polyhistidine tag, a GST fusion, and the like. The expression product optionally includes one or more modified amino acid, such as a glycosylated amino acid, a PEG-ylated amino acid, a farnesylated amino acid, an acetylated amino acid, a biotinylated amino acid, a carboxylated amino acid, a phosphorylated amino acid, an acylated amino acid, or the like.

The method for producing a modified gene construct can further include the step of introducing the modified gene fusion construct into a eukaryotic system. The eukaryotic system can be any of a number of biological systems, including a mammalian system (for example, murine, rodent, guinea pig, rabbit, canine, feline, primate or human systems). Alternatively, the eukaryotic system can be an avian, amphibian, reptilian, or fish system. Preferably, the eukaryotic system is a plant system. A further description of gene expression methodologies is provided below, in the section titled “Expression of Gene Fusion Constructs.”

In embodiments of the invention wherein the eukaryotic system is a plant system the modified gene construct can comprise nucleic acid sequences that are derived from a plant, or nucleic acid sequences that are not derived from a plant (e.g., derived from a bacteria), or some combination plant- and non-plant-derived sequences. For example, some preferred embodiments of the invention involve the introduction of a modified gene construct comprising one or more nucleic acid sequences that are derived from a non-plant microorganism, such as a bacteria or archaea. A potentially powerful application of this approach involves introduction into a plant of a metabolic pathway that does not normally exist in the plant. An example described in more detail below is the introduction of the ectoine synthesis pathway from a halophilic bacteria into plant to increase the stress tolerance of the resulting plant.

A modified gene construct of the invention can comprise two, three, four, five, or more enzymatic domains, wherein one or more of the enzymatic domains has been modified as described herein.

In a preferred embodiment of the invention the modification of a nucleic acid element of a modified gene construct is achieved by shuffling homologous parental sequences (orthologs or paralogs). Parental sequences can be derived from plants or non-plants. The invention includes modified nucleic acids derived from shuffling plant and non-plant derived sequences (e.g., shuffling homologous sequences from plants and bacteria). In some aspects of the invention sequences of low homology or even no discernible homology can be shuffled to arrive at nucleic acids useful in the preparation of a modified gene construct.

Nucleic acid sequences encoding enzymatic domains from any number of metabolic pathways of interest can be incorporated into the modified gene fusion constructs produced by the methods of the present invention. In addition, novel metabolic pathways can be created by the fusion of enzymatic domains which can, in a stepwise manner, use a series of related substrates/intermediates to produce a desired final product.

In one embodiment of the methods for producing a modified gene fusion construct, the enzymatic domains encoded by the two or more nucleic acid sequences are derived from the enzymes phytoene synthase, phytoene desaturase, and/or beta-cyclase. In an alternate embodiment of the present invention, the enzymatic domains encoded by the two or more nucleic acid sequences are derived from the enzymes diaminobutyric acid aminotransferase, diaminobutyric acid acetyltransferase, and ectoine synthase. In another embodiment of the present invention, the enzymatic domains encoded by the two or more nucleic acid sequences are derived from the enzymes beta-ketothiolase, D-reductase, and poly(hydroxyalkanoate) synthase. In a further embodiment of the present invention, the two or more nucleic acid sequences are derived from the following classes of enzymes: ketosynthase-acyltransferases, chain length factors, acyl carrier proteins, and cyclases.

Methods for Producing Gene Fusion Constructs

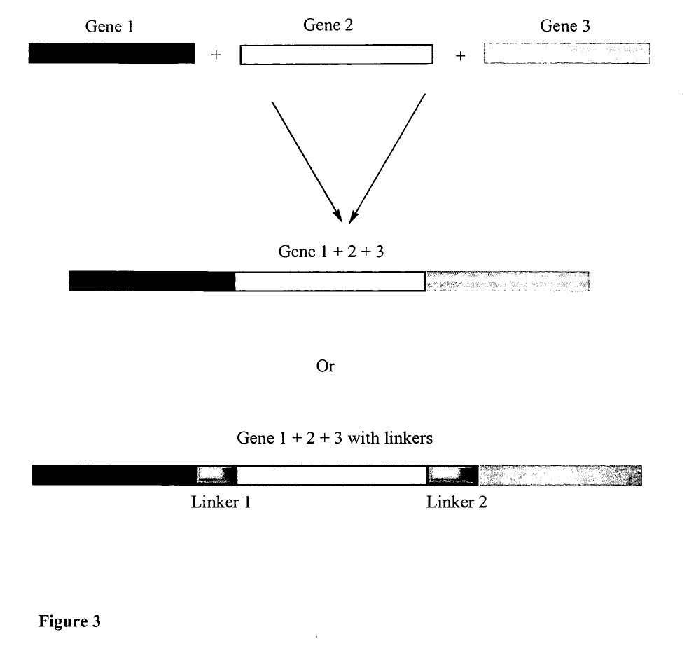

The present invention also provides methods for producing a gene fusion construct by cojoining two or more nucleic acid sequences encoding at least two enzymatic domains that participate in a common metabolic pathway. In some preferred embodiments of the invention, three or more nucleic acid sequences encoding at least two enzymatic domains are cojoined to produce a gene fusion construct. Specific metabolic pathways contemplated for use in the invention include carotenoid biosynthesis, ectoine biosynthesis, polyhydroxyalkanoate biosynthesis, and aromatic polyketide biosynthesis. These pathways and their constituent enzymatic domains are described in more detail below. The nucleic acid sequences of interest in the previously described method can be employed, however, in this embodiment of the invention modification of any of the nucleic acid sequences incorporated into the gene fusion construct is optional. (FIG. 3) In addition, similar nucleotide linker sequences and transcription regulatory elements can be used. The methods for producing a gene fusion construct can further include the step of introducing the modified gene fusion construct into a eukaryotic system such as those described above, for example, a plant system.

In addition, the present invention provides methods for producing a gene fusion construct by cojoining two or more nucleic acid sequences, each encoding at least one enzymatic domain, wherein one or more of the enzymatic domains are derived from plant enzymes or plant systems. Exemplary biosynthetic pathways derived from plant systems include, but are not limited to, enzymes involved in carotenoid biosynthesis. The nucleic acid sequences encoding the plant-derived enzymatic domains can be cojoined directly, or they can be joined via nucleotide linker sequences, and can also include regulatory sequences, as described above. In addition, the nucleic acid sequences, the nucleotide linker sequences, or both, are optionally modified as described previously, thus forming a modified gene fusion construct. The method optionally further comprises introducing the gene fusion construct (or modified gene fusion construct) into an organism, for example, a prokaryotic system or a eukaryotic system. Exemplary prokaryotic and eukaryotic systems are described in the section titled “Expression of Gene Fusion Constructs.”

The present invention further provides for the production of a gene fusion construct comprising two or more nucleic acid sequences, each encoding at least one enzymatic domain, wherein at least one enzymatic domain is derived from a non-plant species, and introducing the construct into a plant. This can be useful for introducing a heterologous metabolic pathways into a plant, e.g, a pathway normally found in a microorganism. Alternatively, this aspect of the invention can be used to introduce a pathway that functions differently than the corresponding endogenous pathway, e.g., by inserting enzymatic activities from a thermophilic organism into a plant, it is possible to generate a metabolic pathway that is activated at high temperature. In some instances it will be desirable to modify a non-plant nucleic acid sequence such that the enzymatic domain encoded thereby is better suited for activity in a plant environment. A non-limiting example would be the modifying the pH dependence of the activity of a bacterial enzyme for optimal activity in a plant system.

Methods for Expressing a Plurality of Enzyme Activities

The present invention also provides methods for expressing a plurality of enzyme activities in a biological system, for example, a eukaryote or a prokaryote. The methods include the steps of providing a gene fusion construct that encodes a single polypeptide having at least three enzymatic domains, and introducing the gene fusion construct into the biological system. The gene fusion construct comprises a cojoined nucleic acid sequence, having at least three nucleic acid sequences encoding at least three enzymatic domains. Alternatively, the gene fusion construct comprises two or more nucleic acid sequences encoding plant-derived enzymatic domains.

Nucleic acid sequences encoding enzymatic domains from any number of metabolic pathways of interest can be incorporated into the gene fusion constructs produced by the methods of the present invention. In addition, novel metabolic pathways can be created by the fusion of enzymatic domains which can, in a stepwise manner, use a series of related substrates/intermediates to produce a desired final product. The enzymatic domains can be derived from a variety of sources, and from a range of biochemical or metabolic pathways. Optionally, the nucleic acid sequences encode proteins that participate in the same metabolic pathway. In one embodiment of the present invention, the enzymatic domains encoded by the three or more nucleic acid sequences are derived from the enzymes phytoene synthase, phytoene desaturase, and/or beta-cyclase. In an alternate embodiment of the present invention, the enzymatic domains encoded by the three or more nucleic acid sequences are derived from the enzymes diaminobutyric acid aminotransferase, diaminobutyric acid acetyltransferase, and ectoine synthase. In another embodiment of the present invention, the enzymatic domains encoded by the three or more nucleic acid sequences are derived from the enzymes beta-ketothiolase, D-reductase, and poly(hydroxyalkanoate) synthase. In a further embodiment of the present invention, the three or more nucleic acid sequences are derived from the following classes of enzymes: ketosynthase-acyltransferases, chain length factors, acyl carrier proteins, and cyclases.

As described above, the nucleic acid sequences employed in the methods of the present invention can be various forms of deoxyribonucleic acid (for example, genomic DNA, cDNA, sense-strand sequences, antisense-strand sequences, recombinant DNA, shuffled DNA, modified DNA, or DNA analogs) or ribonucleic acid (including, but not limited to, genomic RNA, messenger RNA, catalytic RNA, sense-strand sequences, antisense-strand sequences, recombinant RNA, shuffled RNA, modified RNA, or RNA analogs). The nucleic acid sequences encoding the enzymatic domains can be joined directly to one another, or they can be joined via one or more nucleotide linker sequences ranging in length from about three to about three hundred nucleotides. If the nucleotide linker sequence is not to be excised from the nucleic acid transcript prior to translation, it is preferable that the number of nucleotides comprising the linker be present in sets, or increments, of three, such that the translation of the enzymatic domains transcribed past the linker region is not shifted out of the reading frame. Optionally, one or more of the nucleic acid sequences, and/or one or more of the linker sequences, can be mutated or shuffled (either prior to, or after cojoining of the sequences).

The methods of the present invention can further include the step of expressing the gene fusion construct in the biological system, as described below. Furthermore, the present invention provides gene fusion constructs, and transgenic systems, such as transgenic plant systems, as prepared by the methods of the present invention.

Expression of Gene Fusion Constructs

The practice of the methods of the present invention involves the construction of gene fusion constructs as described above, and, in some aspects, the expression of the recombinant nucleic acids in transfected host cells.

The host cell can comprise a eukaryotic system, for example, a eukaryotic cell, a plant cell, an animal cell, a protoplast, or a tissue culture. The host cell optionally comprises a plurality of cells, for example, an organism. Alternatively, the host cell can comprise a prokaryotic system, including, but not limited to, bacteria (i.e., gram positive bacteria, purple bacteria, green sulfur bacteria, green non-sulfur bacteria, cyanobacteria, spirochetes, thermatogales, flavobacteria, and bacteroides) and archaebacteria (i.e., Korarchaeota, Thermoproteus, Pyrodictium, Thermococcales, methanogens, Archaeoglobus, and extreme halophiles). Preferably, the prokaryotic organism comprises one or more bacterial species of agricultural, environmental, industrial, pharmaceutical or clinical interest, including, but not limited to, Escherichia coli, various Streptomyces species, and various Bacillus species.

Introduction of the gene fusion construct into the desired system can be achieved, for example, by techniques such as electroporation, microinjection, particle bombardment, polyethylene glycol-mediated transformation, or Agrobacterium-mediated transformation. The gene fusion construct (or modified gene fusion construct) can optionally be screened prior to introducing the gene fusion construct into the desired system. In embodiments employing libraries of fusion constructs, the constructs can optionally be screened prior to introducing the library of constructs into the desired system.

In certain embodiments of the methods of the present invention, gene fusion constructs and/or modified gene fusion constructs as described above are introduced into plant systems, thereby providing transgenic plants. Methods of transducing plant cells with nucleic acids are generally available. In addition to Berger, Ausubel and Sambrook, useful general references for plant cell cloning, culture and regeneration include Payne, et al., (1992) Plant Cell and Tissue Culture in Liquid Systems (John Wiley & Sons, Inc. New York, N.Y.) and Gamborg and Phillips, eds. (1995) Plant Cell, Tissue and Organ Culture: Fundamental Methods (Springer Lab Manual, Springer-Verlag, Berlin). Cell culture media are described, for example, in Atlas and Parks, eds. (1993) The Handbook of Microbiological Media (CRC Press, Boca Raton, Fla.). Additional information is found in commercial literature such as the “Life Science Research Cell Culture” catalogue (1998) from Sigma-Aldrich, Inc. (St Louis, Mo., “Sigma-LSRCCC”) and, e.g., the “Plant Culture Catalogue” and supplement (1997) also from Sigma-Aldrich (“Sigma-PCCS”).

Gene fusion constructs and modified gene fusion constructs of the present invention can be introduced into the genome of the desired plant host by a variety of conventional techniques. Techniques for transforming a wide variety of higher plant species are well known and described in the technical and scientific literature. See, e.g., Payne, Gamborg, Atlas, Sigma-LSRCCC and Sigma-PCCS, all supra, as well as, e.g., Weising, et al., (1988) Ann Rev Genet 22:421-477.

For example, nucleic acids may be introduced directly into the genomic DNA of a plant cell using techniques such as electroporation and microinjection of plant cell protoplasts, or the gene fusion constructs can be introduced to plant tissue using ballistic methods, such as DNA particle bombardment. Alternatively, the gene fusion constructs may be combined with suitable T-DNA flanking regions and introduced into a conventional Agrobacterium tumefaciens host vector. The virulence functions of the Agrobacterium host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski, et al., (1984) EMBO J. 3:2717-2722. Electroporation techniques are described in Fromm, et al., (1985) Proc Natl Acad Sci USA 82:5824. Ballistic transformation techniques are described in Klein, et al., (1987) Nature 327:70-73; and Weeks, et al., (1993) Plant Physiol. 102:1077-1084.

In one embodiment, Agrobacterium-mediated transformation techniques are used to transfer shuffled coding sequences to transgenic plants. Agrobacterium-mediated transformation is useful primarily in dicots, however, certain monocots can be transformed by Agrobacterium. For instance, Agrobacterium transformation of rice is described by Hiei, et al., (1994) Plant J 6:271-282; U.S. Pat. No. 5,187,073; U.S. Pat. No. 5,591,616; Li, et al., (1991) Science in China 34:54; and Raineri, et al., (1990) Bio/Technology 8:33. In addition, Xu, et al., (1990) Chinese J Bot 2:81 transformed maize, barley, triticale and asparagus by Agrobacterium infection.

In this technique, the ability of the tumor-inducing (Ti) plasmid of A. tumefaciens to integrate into a plant cell genome is used advantageously to co-transfer a nucleic acid of interest into a recombinant plant cell of the present invention. Typically, an expression vector is produced wherein the gene fusion construct (or modified gene fusion construct) of interest is ligated into an autonomously replicating plasmid which also contains T-DNA sequences. T-DNA sequences typically flank the gene fusion construct and comprise the integration sequences of the plasmid. In addition to the gene fusion construct, T-DNA also typically comprises a marker sequence, e.g., antibiotic tolerance genes. The plasmid with the T-DNA and the gene fusion construct are then transfected into Agrobacterium tumefaciens. For effective transformation of plant cells, the A. tumefaciens bacterium also comprises the necessary vir regions on a native Ti plasmid.

In an alternative transformation technique, both the T-DNA sequences as well as the vir sequences are on the same plasmid. For a discussion of A. tumefaciens gene transformation, see, for example, Firoozabady and Kuehnle in the 1995 Springer Lab Manual on plant cell, tissue and organ culture (cited above).

Numerous protocols for establishment of transformable protoplasts from a variety of plant types and subsequent transformation of the cultured protoplasts are available in the art and are incorporated herein by reference. For examples, see, Hashimoto, et al., (1990) Plant Physiol 93:857; Fowke and Constabel (eds.) (1994) Plant Protoplasts; Saunders, et al., (1993) Applications of Plant In Vitro Technology Symposium, UPM 16-18; and Lyznik, et al., (1991) BioTechniques 10:295, each of which is incorporated herein by reference.

In one embodiment of the present invention, transformation of the plant hosts is accomplished using explants prepared from tissues of the desired plants, e.g., leaves. The explants are incubated in a solution of A. tumefaciens at about 0.8×10⁹ to about 1.0×10⁹ cells/mL for a suitable time, typically several seconds. The explants are then cultured for approximately 2 to 3 days on suitable medium.

Transformed plant cells which are derived by any of the above transformation techniques can be cultured to regenerate a whole plant that possesses the transformed genotype and thus the desired phenotype. Such regeneration techniques are performed via manipulation of certain phytohormones in a tissue culture growth medium, typically relying on a biocide and/or herbicide marker which has been introduced together with the desired nucleotide sequences. Plant regeneration from cultured protoplasts is described in Evans, et al., (1983) Protoplasts Isolation and Culture, Handbook of Plant Cell Culture, pp. 124-176 (Macmillian Publishing Company, New York); and Binding (1985) Regeneration of Plants, Plant Protoplasts, pp. 21-73 (CRC Press, Boca Raton, Fla.). Regeneration can also be obtained from and/or performed using plant callus, explants, organs, or parts thereof. Such regeneration techniques are described generally in Klee, et al., (1987) Ann Rev of Plant Phys 38:467-486. See also, Payne, Gamborg, Atlas, Sigma-LSRCCC and Sigma-PCCS, all supra.

After transformation with Agrobacterium, the explants are transferred to selection media. One of skill will realize that the choice of selection media depends on which selectable marker was co-transfected into the explants. After a suitable length of time, transformants will begin to form shoots. After the shoots are about 1 to 2 cm in length, the shoots can be transferred to a suitable root and shoot media. Selection pressure should be maintained once in the root and shoot media.

The transformants develop roots in 1 to about 2 weeks and form plantlets. After the plantlets are from about 3 to about 5 cm in height, they can be placed in sterile soil in fiber pots. Those of skill in the art will realize that different acclimation procedures should be used to obtain transformed plants of different species. In a preferred embodiment, cuttings, as well as somatic embryos of transformed plants, are transferred to medium for establishment of plantlets, after development of a root and shoot. For a description of selection and regeneration of transformed plants, see, Dodds and Roberts (1995) Experiments in Plant Tissue Culture, 3rd Ed. (Cambridge University Press, Cambridge, UK).

Chloroplasts are a site of action for many activities, and, in some instances, a gene fusion construct may be fused to chloroplast transit sequence peptides to facilitate translocation of the gene products into the chloroplasts. In these cases, it can be advantageous to transform the gene fusion construct into chloroplasts of the plant host cells. Numerous methods are available in the art to accomplish chloroplast transformation and expression (see, e.g., Daniell, et al., (1998) Nature Biotechnology 16:346; O'Neill, et al., (1993) The Plant Journal 3:729; Maliga (1993) TIBTECH 11:1). The expression construct typically comprises a transcriptional regulatory sequence functional in plants operably linked to a genr fusion construct. Expression cassettes that are designed to function in chloroplasts include the sequences necessary to ensure expression in chloroplasts. Typically, the coding sequence is flanked by two regions of homology to the chloroplastid genome to effect a homologous recombination with the chloroplast genome; often a selectable marker gene is also present within the flanking plastid DNA sequences to facilitate selection of genetically stable transformed chloroplasts in the resultant transplastonic plant cells (see, e.g., Maliga (1993) and Daniell (1998), and references cited therein).

The transgenic plants of this invention can be characterized either genotypically or phenotypically to determine the presence of the shuffled gene. Genotypic analysis is the determination of the presence or absence of particular genetic material. Phenotypic analysis is the determination of the presence or absence of a phenotypic trait. A phenotypic trait is a physical characteristic of a plant determined by the genetic material of the plant in concert with environmental factors. The presence of gene fusion constructs (or modified gene fusion constructs) can be detected as described in the preceding sections on identification of an optimized shuffled nucleic acid, e.g., by PCR amplification of the genomic DNA of a transgenic plant and hybridization of the genomic DNA with specific labeled probes. The survival of plants on exposure to a selection process where products encoded by the gene fusion construct helps cope with the stress of selection can also be used to monitor incorporation of the gene fusion construct into the plant.

Essentially any plant can be transformed with the gene fusion constructs of the invention. Suitable plants for the transformation and expression of the novel nucleic acids of this invention include agronomically and horticulturally important species. Such species include, but are not restricted to members of the families: Graminae (including corn, rye, triticale, barley, millet, rice, wheat, oat, etc.); Leguminosae (including pea, bean, lentil, peanut, yam bean, cowpea, velvet bean, soybean, clover, alfalfa, lupine, vetch, lotus, sweet clover, wisteria, and sweetpea); Compositae (the largest family of vascular plants, including at least 1,000 genera, including important commercial crops such as sunflower) and Rosaciae (including raspberry, apricot, almond, peach, rose, etc.), as well as nut plants (including, walnut, pecan, hazelnut, etc.), and forest trees (including Pinus, Quercus, Pseudotsuga, Sequoia, Populus,etc.)

Additionally, preferred targets for modification by the nucleic acids of the invention, as well as those specified above, include plants from the genera: Agrostis, Allium, Antirrhinum, Apium, Arabidopsis, Arachis, Asparagus, Atropa, Avena (e.g., oat), Bambusa, Brassica, Bromus, Browaalia, Camellia, Cannabis, Capsicum, Cicer, Chenopodium, Chichorium, Citrus, Coffea, Coix, Cucumis, Curcubita, Cynodon, Dactylis, Datura, Daucus, Digitalis, Dioscorea, Elaeis, Eleusine, Festuca, Fragaria, Geranium, Glycine, Helianthus, Heterocallis, Hevea, Hordeum (e.g., barley), Hyoscyamus, lpomoea, Lactuca, Lens, Lilium, Linum, Lolium, Lotus, Lycopersicon, Majorana, Malus, Mangifera, Manihot, Medicago, Nemesia, Nicotiana, Onobrychis, Oryza (e.g., rice), Panicum, Pelargonium, Pennisetum (e.g., millet), Petunia, Pisum, Phaseolus, Phleum, Poa, Prunus, Ranunculus, Raphanus, Ribes, Ricinus, Rubus, Saccharum, Salpiglossis, Secale (e.g., rye), Senecio, Setaria, Sinapis, Solanum, Sorghum, Stenotaphrum, Theobroma, Trifolium, Trigonella, Triticum (e.g., wheat), Vicia, Vigna, Vitis, Zea (e.g., corn), and the Olyreae, the Pharoideae and many others. As noted, plants in the family Graminae are a particularly preferred target plants for the methods of the invention.

Common crop plants which are targets of the present invention include corn, rice, triticale, rye, cotton, soybean, sorghum, wheat, oat, barley, millet, sunflower, canola, pea, bean, lentil, peanut, yam bean, cowpea, velvet bean, clover, alfalfa, lupine, vetch, lotus, sweet clover, wisteria, sweetpea, tomato, banana and nut plants (e.g., walnut, pecan, etc).

In addition to plants, other eukaryotes such as fungi, flagellates and cilliates, microsporidia, and even animals (i.e., various fishes, birds, reptiles and mammals) can be transformed with the gene fusion constructs and/or modified gene fusion constructs of the present invention. In addition to the references noted throughout, one of skill can find guidance as to animal cell culture in Freshney (1994) Culture of Animal Cells, a Manual of Basic Technique, 3rd Edition (Wiley-Liss, New York) and the references cited therein. See also, Kuchler, et al., (1977) Biochemical Methods in Cell Culture and Virology (Dowden, Hutchinson and Ross, Inc., New York) and Inaba, et al., (1992) J Exp Med 176:1693-1702. Additional information on cell culture is found in Ausubel, Sambrook and Berger, supra. Cell culture media are described in Atlas and Parks, also supra. Generally, one of skill is fully able to transduce cells from animals, plants, fungi, bacteria and other cells using available techniques. Moreover, one of skill can transduce whole organisms with genetic constructs using available techniques.

Alternatively, prokaryotic systems can be transformed with the gene fusion constructs and/or modified gene fusion constructs of the present invention. Optionally, the prokaryotic systems are transformed with constructs comprising at least one plant-derived nucleic acid sequence. Exemplary systems that can be employed in the methods of the present invention include, but are not limited to, bacterial systems (such as those in the genuses Acetobacter, Acetomonas, Actinomyces, Agrobacterium, Bacillus, Bacterium, Bacteroides, Bogoriella, Bordetella, Borrelia, Burkholderia, Campylobacter, Clostridium, Cryobacterium, Diplococcus, Enterobacter, Enterococcus, Erwinia, Erythrobacter, Escherichia, Eubacterium, Flavobacterium, Haemophilus, Halobacillus, Halobacteroides, Helicobacter, Heliobacillus, Heliobacterium, Klebsiella, Lactobacillus, Legionella, Leucobacter, Listeria, Listonella, Methanomonas, Micrococcus, Mycobacterium, Mycoplana, Neisseria, Peptococcus, Proteus, Pseudomonas, Rhizobacter, Rhizomonas, Rhodobacter, Salmonella, Shigella, Sphingomonas, Spirochaeta, Spirosoma, Staphylococcus, Streptobacillus, Streptobacterium, Streptococcus, Streptomyces, Vibrio, and the like) and archaebacterial systems (such as, for example, Korarchaeota, Thermoproteus, Pyrodictium, Thermococcales, Archaeoglobus, methanogens, and extreme halophiles). Preferably, the prokaryotic organism comprises one or more bacterial species of agricultural, environmental, industrial, pharmaceutical or clinical interest, including, but not limited to, Escherichia coli, various Streptomyces species, and various Bacillus species.

Metabolic Pathways and Systems

Carotenoid Biosynthesis

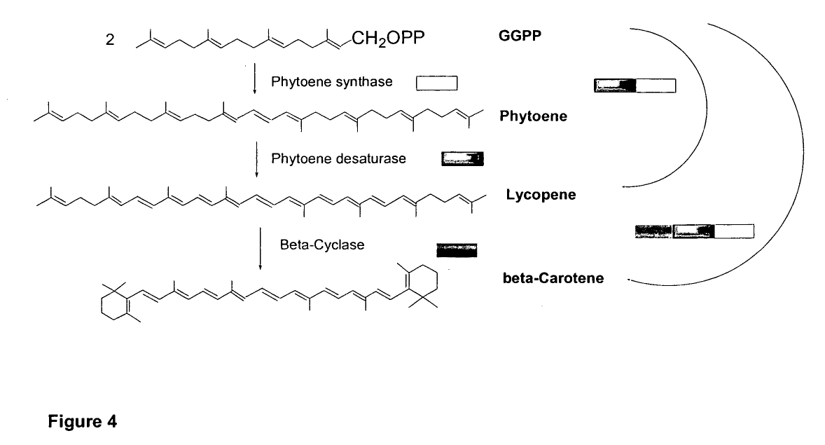

One example of a metabolic system that would be advantageous to express in a eukaryotic system is the metabolism of carotenoids (FIG. 4). Carotenoids are generally colored isoprenoid-based molecules which are synthesized by a variety of plants, molds, yeast, and a few bacteria. In humans, β-carotene functions as a precursor in the synthesis of vitamin A; nutritional deficiencies of β-carotene or vitamin A can lead to susceptibility to infections, night blindness, xerophthalmia (dry eyes), and keratomalacia (excess keratin formation). In addition to the provitamin A, various carotenoids such as lycopene, β-carotene and others are effective antioxidants. Moreover, evidence suggests that carotenoids play an important role in the prevention of cardiovascular disease and cancer (see, for example, Singh and Lippman (1998) “Cancer Chemoprevention, Part 1: Retinoids and Carotenoids and other classic antioxidants” Oncology NY 12:1643-1653). Additional industrial applications of carotenoids include as food colorants, and in animal feeds. In plants, algae, fungi and bacteria, the carotenes more often function in a photosynthetic role.

The biosynthesis of carotenoids is a multistep process involving a series of metabolic enzymes. The starting material in the cell is geranyl geranyl diphosphate (GGPP), a twenty-carbon isoprenoid molecule. Two molecules of GGPP undergo a condensation reaction catalyzed by the enzyme phytoene synthase, to form the 40-carbon intermediate phytoene. In carotenogenic microorganisms, the symmetrical introduction of four double bonds at the C7, C7′, C11 and C11′ positions of the phytoene molecule, via the action of a bacterial phytoene desaturase (also called phytoene dehydrogenase), leads to the next intermediate in the biosynthetic pathway, lycopene. In higher plants, however, formation of lycopene is achieved using two separate enzymes, a plant phytoene desaturase and a z-carotene desaturase. Finally, the enzyme beta-cyclase (also called lycopene cyclase) closes the rings at each end of the lycopene molecule, to form β-carotene. Different cyclases can also be incorporated in the biosynthetic pathway, leading to different cyclization patterns. Further derivations of the carotenoid structure can be achieved by down stream modifying enzymes that exist or are present in various organisms.

Gene fusion constructs and modified gene fusion constructs encoding the β-carotene biosynthetic enzymes (including phytoene synthase, phytoene desaturase, z-carotene desaturase, and lycopene cyclase) as a single nucleic acid transcript would be useful for transformation of eukaryotic systems, such as plant systems. Production of β-carotene in plant systems that already contain the carotenoid metabolic pathway would be enhanced. In addition, plant systems such as rice, and grains which do not naturally synthesize β-carotene, could be enriched nutritionally by the expression of this metabolic pathway. The nucleic acid sequences for these and other carotenoid biosynthetic enzymes can be obtained from GenBank, such as Accession Nos. M84744 (Lycopersicon esculentum), AF220218 (Citrus unshiu), Z37543 (Cucumis melo), X78814 (Narcissus pseudonarcissus), X68017 (Capsicum annuum), AB032797 (Docus carota), U32636 (Zea mays), and additional related sequences, for plant phytoene synthase; Accession Nos. AF195507 (Lycopersicon esculentum), AJ224683 (Narcissus pseudonarcissus), X89897 (Capsicum annuum), AF047490 (Zea mays), and additional related sequences, for plant z-carotene desaturase; Accession Nos. M88683 (Lycopersicon esculentum), X78815 (Narcissus pseudonarcissus), X68058 (Capsicum annuum), U37285 (Zea mays), and additional related sequences, for plant phytoene desaturase; and Accession Nos. X86452 (Lycopersicon esculentum), X86221 (capsicum annuum), U50739 (Arabidopsis thaliana), AF152246 (Citrus x paradisi) and X81787 (Nicotiana tabacum), and additional related sequences, for plant lycopene cyclase (see, WO99/07867 and references cited therein).

In addition, the nucleic acid sequences for carotenoid biosynthetic enzyme clusters from carotenogenic microorganisms can be obtained from GenBank, such as Accession No. M87280 (Erwinia herbicola Eho10), D90087 (Erwinia uredovora), U62808 (Flavobacterium), D58420 (Agrobacterium aurantiacum) and M90698 (Erwinia herbicola Eho13) (and related sequences).

Since most of the carotenoids are colored, desired carotenoid products can be visualized and determined by their characteristic spectra and other analytic methods.

Additional analytical techniques that can be used include, but are not limited to, mass spectrometry, thin layer chromatography (TLC), high pressure liquid chromatography (HPLC), capillary electrophoresis (CE), and NMR spectroscopy.

Ectoine Biosynthesis

Another metabolic system that would be advantageous to produce in eukaryotic systems, particularly plant systems, is the biosynthesis of ectoine (1,4,5,6-tetrahydro-2-methyl-4-pyrimidinecarboxylic acid). Ectoine is a non-toxic, cyclic amino acid, the presence of which has osmoprotective properties, such as conferring increased salt tolerance to cells in vivo. In addition, ectoine appears to protect loss of in vitro activity of various proteins and enzymes placed under stress conditions. Thus, transformation of plant systems with the ectoine biosynthetic machinery would improve the plant's tolerance toward stressful environments (such as high salt, high or low temperatures, drought, and the like). Improved tolerance to these nonideal conditions could result in increased crop productivity. In addition, ectoine can be used as a protein/enzyme stabilizer, or so-called chemical chaperone. Association of enzymes with this chaperone molecule helps to retain the enzymatic activity after repeated freeze/thaw cycles, heat treatment, and/or desiccation. Thus, ectoine also has potential as a stabilizer for use in pharmaceutical, cosmetic, and nutritional compositions.

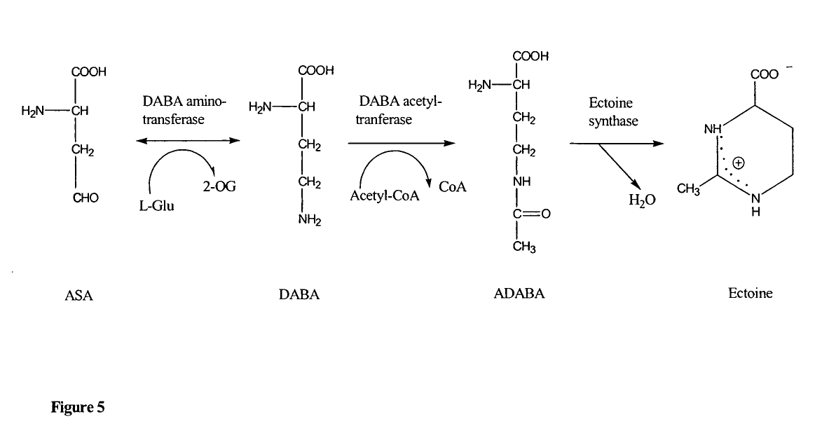

The biosynthesis of ectoine involves three enzymes: diaminobutyric acid aminotransferase (also called a transaminase), diaminobutyric acid acetyltransferase, and ectoine synthase (FIG. 5). In the first reaction of the synthetic pathway, the aminotransferase converts aspartic-semialdehyde and L-glutamine to diaminobutyric acid. Next, the acetyltransferase catalyzes the acetylation of diaminobutyric acid to form N-acetyl diaminobutyric acid. In the final reaction, the N-acetyl diaminobutyric acid is cyclized to produce ectoine via the action of ectoine synthase.

The three genes in the ectoine biosynthetic pathway (etcB, ectA and ectC, respectively) have been isolated from halobacteria. The sequences for these enzymes are available from GenBank, for example, Accession Nos. U66614 (Marinococcus halophilus) and AJ011103 (Halomonas elongata). The optimal pH range for these enzymes is 8.2-9.0, suggesting that some modification to the peptide primary sequence would be desirable prior to expression in a eukaryotic system such as a plant system. This can be achieved, for example, by performing recursive recombination on the nucleic acid sequences encoding these enzymatic domains and incorporation of the modified sequences into a modified gene fusion construct, as described above.

Selection of gene fusion constructs and/or modified gene fusion constructs encoding the enzymes for ectoine biosynthesis can be achieve, for example, by selecting transformed hosts which exhibit an increased tolerance to environmental stress, such as high salt concentrations. For example, wild type E. coli is able to grow at a NaCl concentration up to 3% (0.52 M), while E. coli strains transformed with genes encoding the ectoine biosynthetic pathway, leading to the synthesis of ectoine in vivo, are still viable and able to grow at higher NaCl concentrations, for example 5% NaCl (0.86 M). By growing E. coli transformed with library DNA from gene fusion or shuffling, we will be able to select initial functional clones.

As another example of a selection procedure, yeast can be used to select gene fusion constructs and/or modified gene fusion constructs having desired characteristics. Yeast are viable over a broad range of pH (down to a pH of ˜3) and salt concentrations (up to ˜1M), but a yeast strain with gpd (glycerol phosphate dehydrogenase) knockout is salt-sensitive. Expression of the ectoine biosynthetic pathway, and synthesis of ectoine in gpd knockout yeast recovers (or partially recover) the organism's salt-resistance. However, a gpd deletion strain carrying wild type ectoine biosynthesis pathway enzyme at a low expression level may still not be able to grow at high salt, if the pH of the growth medium is not optimal to the wild type enzyme. Only an ectoine biosynthesis pathway enzyme with an altered optimal pH will be able to produce necessary amount of ectoine product to restore the growth of a salt-sensitive strain. Therefore, a yeast salt-sensitive strain may be used as a host for initial selection for clones with altered optimal pH.

Polyhydroxyalkanoate Biosynthesis

Yet another metabolic pathway that can be incorporated into gene fusion constructs and modified gene fusion constructs of the present invention is the biosynthetic pathway leading to polyhydroxyalkanoates (PHAs). PHAs such as poly-3-hydroxybutyric acid are biodegradable polymers produced as carbon and energy reserves by microorganisms such as Aeromonas, Alcaligenes, Bacillus, Burkholderia, Chromatium, Comamonas, Nocardia, Pseudomonas, Ralstonia, and Rhodospirillum. These biopolymers, which can be formed from a variety of monomeric units, have multiple industrial and medical applications, including production of thermoplastics and drug delivery matrices. The physical and chemical properties of this class of polymer are determined in part by the length of the side chain; polymers having shorter sidechains tend to be semi-crystalline, and are fairly thermoplastic, while polymers having longer sidechains are more elastomeric.

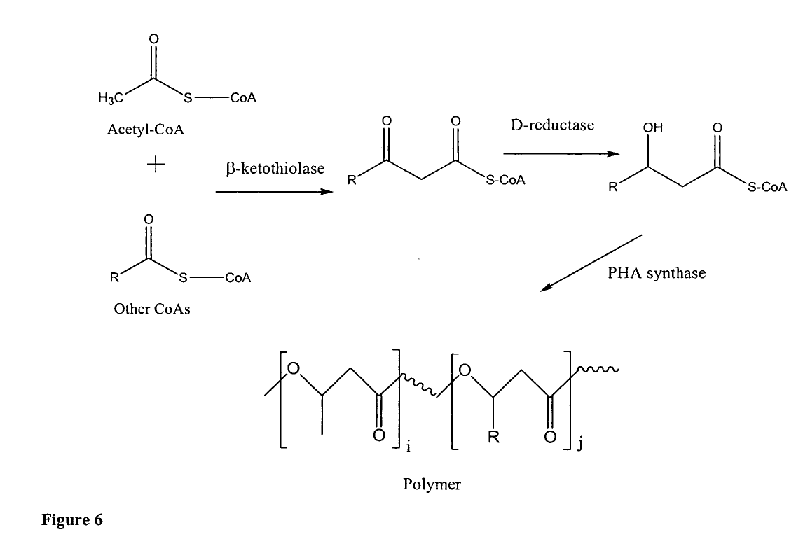

The biosynthesis of short side-chain PHAs involves three enzymes and acetyl-CoA as the starting material (FIG. 6). The first enzyme, a ketothiolase, condensed two building block molecules, such as acetyl CoA molecules, to form an intermediate substrate (acetoacetyl-CoA). The intermediate substrate is subsequently reduced via an NADH- or NADPH-dependent mechanism by a reductase enzyme to form a hydroxyalkanoate-CoA molecule. Finally, the hydroxyalkanoate-CoA is polymerized by a PHA synthase to form the PHA polymer. The PHAs, which can range in size from 10³-10⁸ daltons, are generally stored in granules, or “inclusion bodies” within the cell. Other types of polymers can be generated by starting with building blocks of different lengths and/or compositions. The physical properties of the resulting polymers is influenced, in part, by the length of the side chains incorporated within the final products.

The sequences for these enzymes are available from GenBank, including, but not limited to, Accession Nos. AF153086 (Burkholderia sp DSMZ 9242), U47026 (Alcaligenes latus), AF109909 (Bacillus megaterium), AB009273 (Comamonas acidovorans) and related sequences.

Production of PHAs in cell based systems can be visualized by immunofluorescence with specific chemicals, since PHAs are usually accumulated as granules. Other analytical methods such as NMR spectroscopy (including LC/NMR), mass spectrometry (including techniques and/or instrumentation such as electron ionization, fast atom/ion bombardment, matrix-assisted laser desorption/ionization (MALDI), electrospray ionization, tandem MS, GC/MS, and the like.), high pressure liquid chromatography (HPLC), and capillary electrophoresis (CE), can be used for determination of the polymer composition.

Biosynthesis of Aromatic Polyketides

Further metabolic pathways that could be encoded by the gene fusion constructs or modified gene fusion constructs of the present invention include the minimal aromatic polyketide synthases, which are multienzyme systems that synthesize precursors for a broad range of products, including antibiotics, antifungals, anti-tumor agents, cardiovascular agents, and estrogen receptor antagonists. Examples of aromatic polyketides include, but are not limited to, anthraquinones, doxorubicin, enediyenes, macrolide polyketides such as erythromycin and rifamycin, anthracyclines, nogalamycin, aklavinone and other aclacinomycins; mithramycin and other aureolic acid-based antibiotics.

The minimal polyketide synthase system includes a ketosynthase-acyltransferase, a chain length factor, and an acyl carrier protein. Auxiliary components to this system include a variety of ketoreductases, aromatases and cyclases (see, for example, Carreras, et al., (1997) Topics in Current Chemistry 188:85-126 and references cited therein). Polyketide synthetic machinery has been isolated from a variety of sources, including bacteria, fungi, and plants. While the number of participatory enzymes and the arrangement of the enzymatic domains can differ depending upon the source, the chemical reactions involved in the synthesis of these polymers can be described as follows. The sequences for exemplary polyketide synthesis enzymes are available from GenBank, including, but not limited to, Accession Nos. X63449 (Streptomyces coelicolor), X77865 (Streptomyces griseus), AF126429 (Streptomyces venezuelae), AF098965 (Streptomyces arenae) and related sequences.

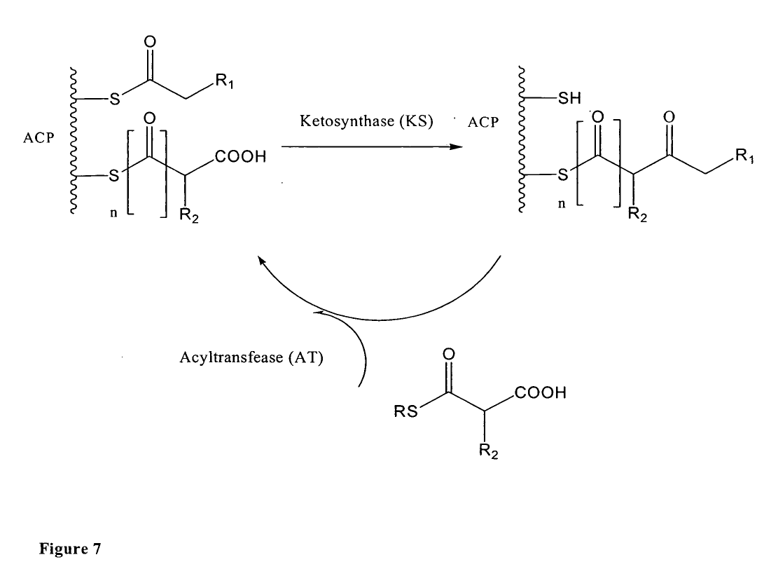

The polyketide metabolic pathway (FIG. 7) starts with a short-chain carboxylic acid “starter unit” such as an acetate or proprionate. Coenzyme A-thioesters of the starter unit are condensed with coenzyme A-thioesters of a dicarboxylic acid “extender group” such as malonate or methyl malonate, via the action of the ketosynthase-acyltransferase. The nascent polyketide chain is retained by the ketosynthase-acyltransferase, while, with each round of condensation/chain elongation, the acyl carrier protein provides further CoA-linked extender groups for addition onto the growing polyketide chain. The chain length factor dictates the length to which the polyketide is elongated. The chain length, extent of ketoreduction (if any), and regiospecificity of cyclization of the final product are all determined by the metabolic enzymes involved in the biosynthesis.

A further modification to the growing polyketide chain can occur, independent of enzyme-based catalysis. Linear polyketide precursors produced by the minimal aromatic polyketide synthases can auto-cyclize to form different types of aromatic polyketides without the presence of the specific cyclase (see, for example, Yuemao Shen, et al., (1999) “Ectopic expression of the minimal whiE polyketide synthase generates a library of aromatic polyketides of diverse sizes and shapes” Proc Natl Acad Sci 96:3622-3627).

The nucleic acid sequences encoding various ketosynthase-acyltransferases and chain length factors are similar in sequence across a number of different species. Shuffling of these sequences provides modified nucleic acid sequences for use in the modified gene fusion constructs of the present invention. Specifically, shuffling the chain length factor can be used to produce enzymes capable of synthesizing novel polyketides, for example, linear aromatic polyketide precursors with varying chain lengths. As an additional source of variation, these enzymatic domains are similar in sequence to fatty acid synthases which could also be used in the generation of nucleotide sequence modifications as described above.

The metabolites and/or products produced by expression of gene fusion constructs and/or modified gene fusion constructs encoding the polyketide biosynthetic machinery can be detected and analyzed by conventional analytic methods and techniques, such as mass spectroscopy, NMR spectroscopy, and the like. Alternatively, the metabolites, or the host cells in which they were synthesized, can be screened for biological activities against interesting targets. For example, aromatic polyketides having antibiotic or other biocide-related activities can be screened against targets, such as pathogenic microorganism, disease associated cell types, or whole animals.

Uses of the Methods and Compositions of the Present Invention

Modifications can be made to the method and materials as described above without departing from the spirit or scope of the invention as claimed, and the invention can be put to a number of different uses, including:

The use of any method herein, to produce any composition or transgenic organism herein.

The use of a method or an integrated system to produce a transgenic organism, for example, a transgenic prokaryote, a transgenic eukaryote, a transgenic plant, and the like.

The use of a method or an integrated system to produce a gene fusion construct or a modified gene fusion construct.

The use of a method or an integrated system to express a plurality of enzymatic activities in a prokaryotic system or a eukaryotic system.

The use of a transgenic organism that has been transformed with one or more gene fusion constructs or modified gene fusion constructs of the present invention, in accordance with the methods described herein as well as those that are known in the art.

In an additional aspect, the present invention provides kits embodying the methods and compositions herein, and utilizing a use of any one or more of the selection strategies, materials, components, methods or substrates hereinbefore described. Kits of the invention optionally comprise one or more of the following: (1) a gene fusion construct or modified gene fusion construct as described herein; (2) instructions for practicing the methods described herein, and/or for operating the selection procedure herein; (3) one or more assay components, including, but not limited to, one or more buffers, enzymes, cofactors, substrates, inhibitors, catalysts, and the like; (4) a container for holding nucleic acids, plants, cells, or the like and, optionally, (5) packaging materials.

In a further aspect, the present invention provides for the use of any component or kit herein, for the practice of any method or assay herein, and/or for the use of any apparatus or kit to practice any assay or method herein.

### EXAMPLES

The following example is offered to illustrate, but not to limit, the claimed invention.

### Example 1

### Preparation and Functional Assessment of a Gene Fusion Construct Encoding Three Enzymatic Domains—Ectoine Synthase

Cloning of Wild-type Ectoine Synthase Operon

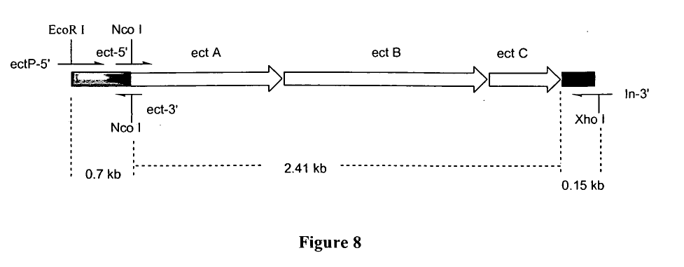

Marinococcus halophilus (ATCC 27964) containing the ectoine synthase operon of interest was obtained from ATCC. The operon, which includes the ect A (diaminobutyric acid acetyltransferase), ect B (diaminobutyric acid aminotransferase) and ect C (ectoine synthase) genes, has been characterized and is available at GenBank (Accession No. U66614). The 3.26 kb operon, extending from 0.7 kb upstream of the ect A start codon to 0.15 kb downstream of the ect C stop codon, was amplified from genomic DNA using Herculase enhanced DNA polymerase (Stratagene) (FIG. 8) and the following primer pairs: ectP-5′ (5′-TAAGAATTCGGGTAGTACACGCAAGGATGGG-3′ (SEQ ID NO: 1; EcoR I site is underlined)) and ect-3′ (5′-CGTTTCCATGGTCTTACCACCTTTTAAAAGTMTAG-3′ (SEQ ID NO: 2; Nco I site is underlined)) was used to PCR out a 0.7 kb fragment, with introduction of EcoR I and Nco I sites; ect-5′ (5′-AGGTGGTAAGACCATGGAAACGAAAATGACTGGAACG-3′ (SEQ ID NO: 3; Nco I site is underlined)) and In-3′ (5′-AGGAGAAACTCGAGACTTCGCGCTTTACTTCTTCCGG-3′ (SEQ ID NO: 4; XhoI site is underlined)) was used to PCR out a 2.56 kb fragment, with introduction of Nco I and Xho I sites.

E. coli vector pBR322 was digested by EcoR I and Sal I (compatible end with Xho I) restriction enzymes to create a cloning vector for EcoR I/Nco I (0.7 kb) and Nco I/Xho I (2.56 kb) fragments obtained above. After three fragment ligation, it was transformed into Top10 E. coli competent cells to form the pBR322-wt construct.

Preparation of an Ectoine Synthase Gene Fusion Construct

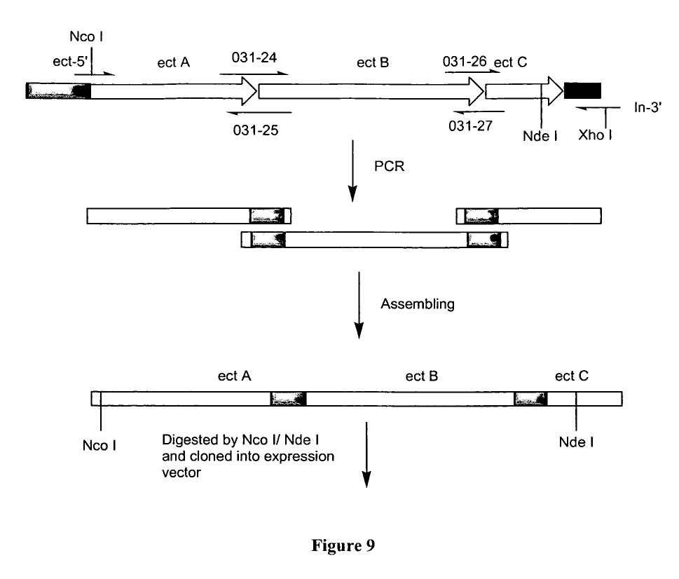

The ect A, ect B and ect C genes were combined to form a gene fusion construct (FIG. 9). The process entailed removing ect A and ect B stop codons, inter-gene spaces and ect B and ect C start codons, and fusing ect A, ect B and ect C in-frame with four-glycine linker sequences. The construction was accomplished using the following PCR operations: an ect A fragment was generated using the primer pair ect-5′ (SEQ ID NO: 3) and 031-25 (5′-CGCTGAGATCATTCTGGCCACCGCCACCCTTTGTAAATGGTCCTATTCGAAATG TC-3′ (SEQ ID NO: 5; site encoding the 4-glycine linker is underlined)); an ect B fragment was generated using the primer pair 031-24 (5′-CCATTTACAAAGGGTGGCGGTGGCCAGAATGATCTCAGCGTTTTTAATGAATAC G-3′ (SEQ ID NO: 6; site encoding the 4-glycine linker is underlined)) and 031-27 (5′-GTTTAATTACTTTACCGCCACCGCCTTTGGCTACGAGGTTGCTTTCAGCGGTAA C-3′ (SEQ ID NO: 7; site encoding the 4-glycine linker is underlined)); and an ect C fragment was generated using the primer pair 031-26 (5′-CCTCGTAGCCAAAGGCGGTGGCGGTAAAGTAATTAAACTCGAAGATTTGCTCG GC-3′ (SEQ ID NO: 8; site encoding the 4-glycine linker is underlined)) and In-3′ (SEQ ID NO: 4).

The three overlapping PCR fragments were assembled by 5-10 PCR cycles without primer at a condition of 95° C. melting temperature for 30 sec, 60° C. annealing temperature for 30 sec and 72° C. extension temperature for 1 min. The assembled product was then amplified with the primers of ect-5′ and In-3′ at an annealing temperature of 55° C. The Herculase enhanced DNA polymerase (Stratagene) was used for both assembly and subsequent PCR amplification. The resulting PCR product was digested with Nco I and Nde I restriction enzymes and cloned into Nco I/Nde 1-digested pBR322. The linker regions of the construct were confirmed by sequencing analysis. The resulting plasmid was transformed into Top10 E. coli competent cells.

Assessment of the Ability of E. coli Transformed with the Ectoine Synthase Gene Fusion Construct to Tolerate Salt

Top 10′ cells transformed with the wt ectoine operon and with the ectoine synthase fusion construct were tested for the ability to grow at various salt concentrations. The test involved growing the cells at 37° C. for 36 hours in the following medium: MM63 (100 mM KH₂PO₄, 75 mM KOH, 15 mM (NH₄)₂SO₄, 1 mM MgSO₄, 3.9 μM FeSO₄, 22 mM glucose, 1.5 ml/l vitamin solution, pH7.4. (vitamin solution: 10 mg biotin, 35 mg nicotinamide, 30 mg thiamine dichloride, 20 mg p-aminobenzoic acid, 10 mg pyridoxal chloride, 10 mg Ca-pantothenate, 5 mg vitamine B12 in 100 ml H₂O)) plus 10% LB and varying amounts of salt (0-5% NaCl). The cell density of the culture was measured by spectrophotometer at 600 nm.

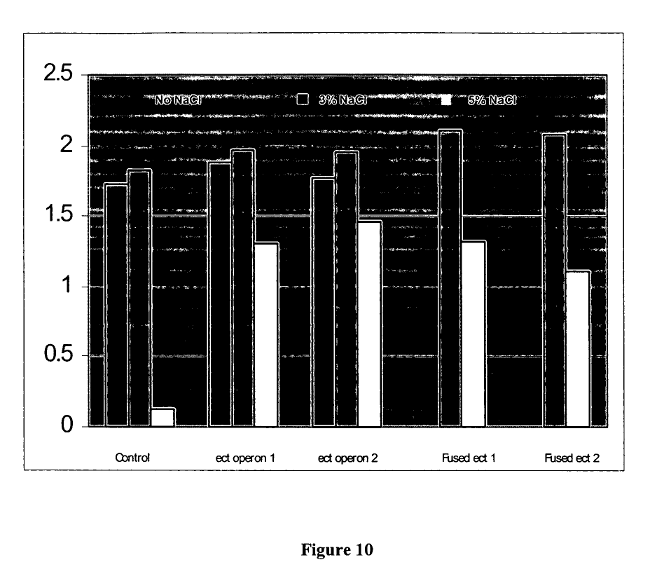

The results (FIG. 10) show that the three gene fusion construct confers upon E. coli an ability to tolerate salt comparable to the wild-type ectoine operon.

While the foregoing invention has been described in some detail for purposes of clarity and understanding, it will be clear to one skilled in the art from a reading of this disclosure that various changes in form and detail can be made without departing from the true scope of the invention. For example, all the techniques and, compositions described above may be used in various combinations. All publications, patent documents (e.g., applications, patents, etc.) or other references cited in this application are incorporated by reference in their entirety for all purposes to the same extent as if each individual publication or patent document were individually so denoted.

What is claimed is:

1. A method of producing a modified gene fusion construct, the method comprising cojoining two or more heterologous nucleic acid sequences, wherein each heterologous nucleic acid sequence encodes one or more enzymatic domains, and wherein at least one of the two or more heterologous nucleic acid sequences is modified, thereby producing the modified gene fusion construct.

2. The method of claim 1, wherein at least one of the two or more nucleic acid sequences is modified prior to cojoining the two or more nucleic acid sequences.

3. The method of claim 1, wherein at least one of the two or more nucleic acid sequences is modified after cojoining the two or more nucleic acid sequences.

4. The method of claim 1, wherein the one or more enzymatic domains participate in the same metabolic pathway.

5. The method of claim 1, wherein at least one of the two or more nucleic acid sequences is modified by shuffling at least one nucleic acid sequence.

6. The method of claim 5, wherein shuffling the at least one nucleic acid sequence comprises recursive sequence recombination.

7. The method of claim 1, wherein the two or more nucleic acid sequences encode enzymatic domains selected from the group consisting of phytoene synthase, phytoene desaturase, z-carotene desaturase, and beta-cyclase.

8. The method of claim 1, wherein the two or more nucleic acid sequences encode enzymatic domains selected from the group consisting of diaminobutyric acid aminotransferase, diaminobutyric acid acetyltransferase, and ectoine synthase.

9. The method of claim 1, wherein the two or more nucleic acid sequences encode enzymatic domains selected from the group consisting of beta-ketothiolase, D-reductase, and poly(hydroxyalkanoate) synthase.

10. The method of claim 1, wherein the two or more nucleic acid sequences encode enzymatic domains selected from the group consisting of a ketosynthase-acyltransferase, a chain length factor, an acyl carrier protein, and a cyclase.

11. The method of claim 1, wherein cojoining the two or more nucleic acid sequences comprises connecting the two or more nucleic acid sequences directly to one another.

12. The method of claim 1, wherein cojoining the two or more nucleic acid sequences comprises connecting the two or more nucleic acid sequences with one or more nucleotide linker sequences.

13. The method of claim 12, wherein the one or more nucleotide linker sequences independently comprise between about three and about 300 nucleotides.

14. The method of claim 13, wherein the one or more nucleotide linker sequences independently comprise between about 12 to about 90 nucleotides.

15. The method of claim 12, wherein at least one of the one or more nucleotide linker sequences comprises one or more intron sequences.

16. The method of claim 12, wherein at least one of the one or more nucleotide linker sequences comprises a nucleotide sequence that encodes a peptide linker.

17. The method of claim 16, wherein the peptide linker comprises a cleavable peptide sequence or an intein sequence.

18. The method of claim 16, wherein at least about 80% of the amino acid residues in the peptide linker are selected from the group consisting of alanine and glycine residues.

19. The method of claim 1, wherein the modified gene fusion construct comprises one or more transcription regulatory sequences.

20. The method of claim 19, wherein the one or more transcription regulatory sequences comprises one or more plant transcription regulatory sequences.

21. The method of claim 1, further comprising introducing the modified gene fusion construct into a eukaryotic system.

22. The method of claim 21, wherein the eukaryotic system is a plant system.

23. The method of claim 22, wherein the plant system is selected from Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Hyoscyamus, Lycopersicon, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, Malus, Apium, Narcissus, Docus, and Datura.

24. A transgenic plant prepared by the method of claim 22.

25. A modified gene fusion construct comprising two or more cojoined heterologous nucleic acid sequences, wherein each nucleic acid sequence encodes one or more enzymatic domains, and wherein at least one of the two or more nucleic acid sequences is modified.

26. The modified gene construct of claim 25, wherein at least one of the two or more heterologous nucleic acid sequences is shuffled.

27. The modified gene fusion construct of claim 25, wherein the two or more heterologous nucleic acid sequences encode at least two enzymatic domains selected from the group consisting of phytoene synthase, phytoene desaturase, z-carotene desaturase, and beta-cyclase.

28. The modified gene fusion construct of claim 25, wherein the two or more nucleic acid sequences encode at least two enzymatic domains selected from the group consisting of diaminobutyric acid aminotransferase, diaminobutyric acid acetyltransferase, and ectoine synthase.

29. The modified gene fusion construct of claim 25, wherein the two or more nucleic acid sequences encode at least two enzymatic domains selected from the group consisting of beta-ketothiolase, D-reductase, and poly(hydroxyalkanoate) synthase.

30. The modified gene fusion construct of claim 25, wherein the two or more nucleic acid sequences encode at least two enzymatic domains selected from the group consisting of a ketosynthase-acyltransferase, a chain length factor, an acyl carrier protein, and a cyclase.

31. A vector comprising the modified gene fusion construct of claim 25 and a promoter.

32. A method of producing a gene fusion construct, the method comprising cojoining two or more heterologous nucleic acid sequences that participate in the same metabolic pathway, wherein at least one of the cojoined nucleic acid sequences is derived from a eukaryote and another cojoined nucleic acid sequence is derived from either a different species of eukaryote or from a prokaryote.

33. The method of claim 32, wherein at least one of the cojoined nucleic acid sequences is derived from a plant.

34. The method of claim 32, wherein at least one of the cojoined nucleic acid sequences is derived from a prokaryote.

35. The method of claim 34, wherein at least one of the cojoined nucleic acid sequences is derived from a plant.

36. The method of claim 32, wherein at least two of the cojoined nucleic acid sequences are derived from different plant species.

37. The method of claim 32, wherein the method comprises cojoining three or more heterologous nucleic acid sequences that participate in the same metabolic pathway.

38. The method of claim 32, wherein at least one of the heterologous nucleic acid sequences is modified.

39. The method of claim 38, wherein at least one of the heterologous nucleic acid sequences is shuffled.

40. The method of claim 32, wherein the two or more nucleic acid sequences encode enzymatic domains selected from the group consisting of phytoene synthase, phytoene desaturase, z-carotene desaturase, and beta-cyclase.

41. The method of claim 32, wherein the two or more nucleic acid sequences encode enzymatic domains selected from the group consisting of diaminobutyric acid aminotransferase, diaminobutyric acid acetyltransferase, and ectoine synthase.

42. The method of claim 32, wherein the two or more nucleic acid sequences encode enzymatic domains selected from the group consisting of beta-ketothiolase, D-reductase, and poly(hydroxyalkanoate) synthase.

43. The method of claim 32, wherein the two or more nucleic acid sequences encode enzymatic domains selected from the group consisting of a ketosynthase-acyltransferase, a chain length factor, an acyl carrier protein, and a cyclase.

44. The method of claim 32, wherein the cojoined nucleic acid sequences are connected directly to one another.

45. The method of claim 32, wherein the cojoined nucleic acid sequences are connected to one another with one or more nucleotide linker sequences.

46. The method of claim 32, wherein the modified gene fusion construct comprises one or more transcription regulatory sequences.

47. The method of claim 46, wherein the one or more transcription regulatory sequences comprises one or more plant transcription regulatory sequences.

48. The method of claim 32, further comprising introducing the modified gene fusion construct into a eukaryotic system.

49. The method of claim 48, wherein the eukaryotic system is a plant system.

50. The method of claim 49, wherein the plant system is selected from Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Hyoscyamus, Lycopersicon, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, Malus, Apium, Narcissus, Docus, and Datura.

51. A transgenic plant prepared by the method of claim 49.

52. A gene fusion construct comprising two or more cojoined heterologous nucleic acid sequences that participate in the same metabolic pathway, wherein at least one of the cojoined nucleic acid sequences is derived from a eukaryote and another cojoined nucleic acid sequence is derived from either a different species of eukaryote or from a prokaryote.

53. The gene construct of claim 52, wherein at least one of the nucleic acid sequences is modified.

54. The gene construct of claim 53, wherein at least one of the nucleic acid sequences is shuffled.

55. The gene fusion construct of claim 52, wherein the two or more heterologous nucleic acid sequences encode at least two enzymatic domains selected from the group consisting of phytoene synthase, phytoene desaturase, z-carotene desaturase, and beta-cyclase.

56. The gene fusion construct of claim 52, wherein the two or more nucleic acid sequences encode at least two enzymatic domains selected from the group consisting of diaminobutyric acid aminotransferase, diaminobutyric acid acetyltransferase, and ectoine synthase.

57. The gene fusion construct of claim 52, wherein the two or more nucleic acid sequences encode at least two enzymatic domains selected from the group consisting of beta-ketothiolase, D-reductase, and poly(hydroxyalkanoate) synthase.

58. The gene fusion construct of claim 52, wherein the two or more nucleic acid sequences encode at least two enzymatic domains selected from the group consisting of a ketosynthase-acyltransferase, a chain length factor, an acyl carrier protein, and a cyclase.

59. A vector comprising the modified gene fusion construct of claim 52 and a promoter.

60. A gene fusion construct comprising two or more cojoined heterologous nucleic acid sequences, wherein the two or more nucleic acid sequences encode at least two enzymatic domains selected from the group consisting of diaminobutyric acid aminotransferase, diaminobutyric acid acetyltransferase, and ectoine synthase.

61. The recombinant nucleic acid sequence of claim 60, wherein at least one of the at least two cojoined nucleic acid sequences is modified.

62. The recombinant nucleic acid sequence of claim 61, wherein at least one of the at least two cojoined nucleic acid sequences is shuffled.

63. A gene fusion construct comprising three or more cojoined heterologous nucleic acid sequences, wherein the three or more nucleic acid sequences encode at least three enzymatic domains selected from the group consisting of beta-ketothiolase, D-reductase, and poly(hydroxyalkanoate) synthase.

64. The recombinant nucleic acid sequence of claim 63, wherein at least one of the at least three cojoined nucleic acid sequences is modified.

65. The recombinant nucleic acid sequence of claim 64, wherein at least one of the at least three cojoined nucleic acid sequences is shuffled.

66. A gene fusion construct comprising two or more cojoined heterologous nucleic acid sequences, wherein the two or more nucleic acid sequences encode at least two enzymatic domains selected from the group consisting of a ketosynthase-acyltransferase, a chain length factor, an acyl carrier protein, and a cyclase.

67. The recombinant nucleic acid sequence of claim 66, wherein at least one of the at least two cojoined nucleic acid sequences is modified.

68. The recombinant nucleic acid sequence of claim 67, wherein at least one of the at least two cojoined nucleic acid sequences is shuffled.

69. A gene fusion construct comprising two or more cojoined heterologous nucleic acid sequences, wherein the two or more nucleic acid sequences encode at least two enzymatic domains selected from the group consisting of a phytoene synthase, phytoene desaturase, z-carotene desaturase, and beta-cyclase.

70. The recombinant nucleic acid sequence of claim 69, wherein at least one of the at least two cojoined nucleic acid sequences is modified.

71. The recombinant nucleic acid sequence of claim 70, wherein at least one of the at least two cojoined nucleic acid sequences is shuffled.

72. A hybrid protein comprising two or more heterologous enzymatic domains that participate in the same metabolic pathway, wherein at least one of the two or more enzymatic domains is encoded by a nucleic acid sequence that has been modified.

73. The hybrid protein of claim 72, wherein at least one of the two or more enzymatic domains is encoded by a nucleic acid sequence that has been shuffled.

74. The hybrid protein of claim 72, wherein two or more enzymatic domains are connected by one or more peptide linker sequences.

75. The hybrid protein of claim 74, wherein at least one of the one or more peptide linker sequences comprises a cleavable peptide sequence or an intein sequence.

76. The hybrid protein of claim 72, wherein the enzymatic domains that participate in the same metabolic pathway comprise one or more of phytoene synthase, phytoene desaturase, z-carotene desaturase, or beta-cyclase.

77. The hybrid protein of claim 72, wherein the enzymatic domains that participate in the same metabolic pathway comprise one or more of diaminobutyric acid aminotransferase, diaminobutyric acid acetyltransferase, or ectoine synthase.

78. The hybrid protein of claim 72, wherein the enzymatic domains that participate in the same metabolic pathway comprise one or more of beta-ketothiolase, D-reductase, or poly(hydroxyalkanoate) synthase.

79. The hybrid protein of claim 72, wherein the enzymatic domains that participate in the same metabolic pathway comprise one or more of a ketosynthase-acyltransferase, a chain length factor, an acyl carrier protein, or a cyclase.

80. A method of producing a gene fusion construct, the method comprising cojoining two or more nucleic acid sequences encoding at least two enzymatic domains, wherein at least one of the nucleic acid is derived from a plant, thereby producing a gene fusion construct.

81. The method of claim 80, wherein the plant enzymes are selected from the group consisting of phytoene synthase, phytoene desaturase, z-carotene desaturase, and beta-cyclase.

82. The method of claim 80, wherein the one or more enzymatic domains participate in the same metabolic pathway.

83. The method of claim 80, wherein at least one of the two or more nucleic acid sequences is modified.

84. The method of claim 83, wherein at least one of the two or more nucleic acid sequences is modified by shuffling.

85. The method of claim 84, wherein shuffling the at least one nucleic acid sequence comprises recursive sequence recombination.

86. The method of claim 80, wherein cojoining the two or more nucleic acid sequences comprises connecting the two or more nucleic acid sequences directly to one another.

87. The method of claim 80, wherein cojoining the two or more nucleic acid sequences comprises connecting the two or more nucleic acid sequences with one or more nucleotide linker sequences.

88. The method of claim 87, wherein the one or more nucleotide linker sequences independently comprise between about three and about 300 nucleotides.

89. The method of claim 88, wherein the one or more nucleotide linker sequences independently comprise between about 12 to about 90 nucleotides.

90. The method of claim 87, wherein at least one of the one or more nucleotide linker sequences comprises one or more intron sequences.

91. The method of claim 87, wherein at least one of the one or more nucleotide linker sequences comprises a nucleotide sequence that encodes a peptide linker.

92. The method of claim 91, wherein the peptide linker comprises a cleavable peptide sequence or an intein sequence.

93. The method of claim 91, wherein at least about 80% of the amino acid residues in the peptide linker are selected from the group consisting of alanine and glycine residues.

94. The method of claim 80, wherein the modified gene fusion construct comprises one or more transcription regulatory sequences.

95. The method of claim 94, wherein the one or more transcription regulatory sequences comprises one or more plant transcription regulatory sequences.

96. The method of claim 80, further comprising introducing the modified gene fusion construct into a eukaryotic system.

97. The method of claim 96, wherein the eukaryotic system is a plant system.

98. The method of claim 97, wherein the plant system is selected from Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Hyoscyamus, Lycopersicon, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, Malus, Apium, Narcissus, Docus, and Datura.

99. A transgenic plant prepared by the method of claim 97.

100. A gene fusion construct comprising two or more cojoined heterologous nucleic acid sequences, wherein each nucleic acid sequence encodes one or more enzymatic domains, and wherein at least one of the two or more nucleic acid sequences is derived from a plant.

101. The gene fusion construct of claim 100, wherein at least one of the two or more heterologous nucleic acid sequences is modified.

102. The gene fusion construct of claim 101, wherein at least one of the two or more heterologous nucleic acid sequences is shuffled.

103. The gene fusion construct of claim 100, wherein the two or more heterologous nucleic acid sequences encode at least two enzymatic domains selected from the group consisting of phytoene synthase, phytoene desaturase, z-carotene desaturase, and beta-cyclase.

104. A vector comprising the gene fusion construct of claim 100 and a promoter.


SEQUENCE LISTING (8 sequences)

SEQ ID NO: 1 - DNA, 31 bp, Artificial Sequence
  primer
taagaattcgggtagtacacgcaaggatggg

SEQ ID NO: 2 - DNA, 36 bp, Artificial Sequence
  primer
cgtttggatggtcttaccaccttttaaaagtaatag

SEQ ID NO: 3 - DNA, 37 bp, Artificial Sequence
  primer
aggtggtaagaccatggaaacgaaaatgactggaacg

SEQ ID NO: 4 - DNA, 37 bp, Artificial Sequence
  primer
aggagaaactcgagacttcgcgctttacttcttccgg

SEQ ID NO: 5 - DNA, 56 bp, Artificial Sequence
  primer
cgctgagatcattctggccaccgccaccctttgtaaatggtcctattcgaaatgtc

SEQ ID NO: 6 - DNA, 55 bp, Artificial Sequence
  primer
ccatttacaaagggtggcggtggccagaatgatctcagcgtttttaatgaatacg

SEQ ID NO: 7 - DNA, 55 bp, Artificial Sequence
  primer
gtttaattactttaccgccaccgcctttggctacgaggttgctttcagcggtaac

SEQ ID NO: 8 - DNA, 55 bp, Artificial Sequence
  primer
cctcgtagccaaaggcggtggcggtaaagtaattaaactcgaagatttgctcggc